(19) United States
(12) Patent Application Publication
(10) Pub. No.: US 2005/0244892 A1
(43) Pub. Date: Nov. 3, 2005

(54) Resistin as a marker and therapeutic target for cardiovascular disease

(76) Inventors: Mitchell A. Lazar, Gladwyne, PA (US); Daniel J. Rader, Philadelphia, PA (US); Muredach P. Reilly, Philadelphia, PA (US)

Correspondence Address: HOWSON AND HOWSON;ONE SPRING HOUSE CORPORATION CENTER, BOX 457, 321 NORRISTOWN ROAD, SPRING HOUSE, PA, 19477, US

(21) Appl. No.: 11/068,806

(22) Filed: Feb. 28, 2005

Related U.S. Application Data
(60) Provisional application No. 60/548,795, filed on Feb. 27, 2004.

(51) Int. Cl. G01N 33/53

(52) U.S. Cl. 435/7.1

(57) ABSTRACT

The risk or progression of cardiovascular disease and coronary artery disease is assessed in a mammalian subject by measuring the level or concentration of circulating serum resistin in a subject and comparing the measured level to resistin levels within a standardized or standard population. Methods of treating cardiovascular diseases and/or inflammatory disorders involve administering to a patient a composition that can reduce the circulating levels of resistin.

### CROSS-REFERENCE TO OTHER APPLICATIONS

This application claims the benefit of the priority date of United States Provisional Patent Application No. 60/548,795, filed Feb. 27, 2004. The disclosure of said provisional application is incorporated herein by reference.

### STATEMENT REGARDING FEDERALLY SPONSORED RESEARCH OR DEVELOPMENT

This invention was funded in part by grants from the National Institutes of Health, Nos. 01-RR00040, K23 RR15532-04, R01 HL73278-01, R01 DK49780 and R01 DK49210. The United States government has an interest in this invention.

### BACKGROUND OF THE INVENTION

Dietary and lifestyle changes during the last century have entailed an unprecedented epidemic of obesity and associated metabolic diseases, including type 2 diabetes (Ogden C L et al, 2003, Endocrin. Metab. Clin. North Am., 32:741-760 vii). Additionally, other cardiovascular diseases, such as atherosclerosis, are also on the rise in the population at large, and occur even in the absence of any symptoms or risk factors (Bassuk et al 2004 Curr. Probl. Cardiol., 29:439-493). The convergence of insulin resistance and inflammation in the pathogenesis of atherosclerotic cardiovascular disease (CVD) has been recognized over the past decade. Metabolic syndrome definitions and markers of inflammation, such as C reactive protein, have been proposed for use in clinical practice to aid in the identification of asymptomatic patients at high-risk for CVD. However, there remains uncertainty as to the most appropriate definition of metabolic syndrome and the optimal inflammatory marker for use in clinical practice.

Many individuals suffer simultaneously from several of the above-mentioned conditions, and epidemiological studies in humans, as well as animal models, suggest that obesity-related insulin resistance is a common pathogenic feature (Flier, J S 2004 Cell 116:337-350). Indeed, insulin resistance is the keystone of the “metabolic syndrome”, a major cardiovascular risk factor even in the absence of demonstrable glucose intolerance or diabetes (Sowers and Frohlich, 2004, Med. Clin. North. Am. 88:63-82). Obesity, the most common cause of insulin resistance, and insulin resistance are strongly associated with systemic markers of inflammation and, indeed, inflammation may contribute to insulin resistance (Haffner 2003 Am. J. Cardiol., 92:18J-26J). Obesity is therefore increasingly recognized as a low-grade inflammatory state. Atherosclerosis is similarly increasingly viewed as an inflammatory state.

Similarities and overlap between obesity and inflammatory states are emerging. Inflammatory cytokines such as tumor necrosis factor a (TNFα) and interleukin-6 (IL-6) are produced by adipocytes as well as by monocytes and macrophages, and circulate at increased levels in obesity. Moreover, bone marrow-derived macrophages home to adipose tissue in obesity, and adipocytes and macrophages may even be interconvertible. Furthermore, inflammation is increasingly recognized as a major component and predictor of atherosclerotic vascular disease, a major clinical consequence of insulin resistance (Glass and Witztum 2001 Cell, 104:503-516). Thus, biomarkers that integrate metabolic and inflammatory signals are attractive candidates for defining risk of atherosclerotic cardiovascular disease (CVD) (Rajala et al, 2003a Endocrinol., 144:3765-73).

Resistin, originally identified and characterized as a circulating mouse adipocyte gene product that is regulated by antidiabetic drugs, belongs to a family of cysteine-rich secretory proteins called resistin-like molecules (RELMs) (Steppan et al, 2001a Nature, 409:307-12; Steppan et al 1, 2001b Proc. Natl. Acad., Sci., USA, 98:502-506) or FIZZ (found in inflammatory zones) proteins (Holcomb et al, 2000 EMBO J., 19:4046-55). In rodents, resistin is almost exclusively derived from fat tissue and adipose expression and serum levels are elevated in models of obesity and insulin resistance (Steppan et al, 2001a, cited above; Kim et al, 2001 J. Biol. Chem., 276:11252-6; and Rajalaetal, 2004 Diabetes, 53:1671-9). Hyperresistinemia impairs glucose tolerance (Steppan et al 1, 2001a, cited above) and induces hepatic insulin resistance in rodents (Rajala et al, 2003b J. Clin. Invest., 111:225-30), whereas mice deficient in resistin are protected from obesity-associated insulin resistance (Banerjee et al, 2004 Science, 303:1195-8).

A syngenic gene exists in humans, but is expressed at much higher levels in the human inflammatory cells, monocytes and macrophages, than in adipocytes (Savage et al, 2001 Diabetes, 50:2199-2202; Patel et al, 2003 Biochem. Biophys Res. Commun., 300:472-6), raising questions about the relationship between resistin and human metabolic disease. Although resistin mRNA is detectable in human adipocytes, levels are much higher in human inflammatory cells. Although assays for human resistin are in their infancy, in the past year several small studies have reported that circulating resistin levels are increased in human obesity (Yannakoulia et al, 2003 J. Clin. Endocrinol. Metab., 88:1730-6; Azuma et al, 2003 Obes. Res., 11:997-1001; Degawa-Yamauchi et al, 2003 J. Clin. Endocrinol. Metab., 88:5452-5; Volarova de Courten et al, 2004 Diabetes, 53:1279-84) and diabetes (McTernan et al, 2003 J. Clin. Endocrinol. Metab., 88:6098-106; Silha et al, 2003 Eur. J. Endocrinol., 149:33105; Youn et al, 2004 J. Clin. Endocrinol. Metab., 89:150-6; Fujinami et al, 2004 Clin. Chim Acta, 339:57-63; Bajaj et al, 2004 Int. J. Obes. Relat. Metab. Disord., 28:783-9). Not all reports have been consistent in this regard (Pfutzner et al, 2003 Clin. Lab., 49:571-6; Hegele et al, 2003 Arterioscler. Thromb. Vasc. Biol., 23:111-6; Lee et al, 2003 J. Clin. Endocrinol. Metab., 88:4848-56; Fehmann and Heyn, 2002 Horm. Metab. Res., 34:671-3). In contrast to rodents, in humans resistin is primarily expressed in inflammatory cells (Fain et al, 2003 Biochem. Biophys. Res. Commun. 300:674-8;Yang et al, 2003 Biochem. Biophys Res. Commun., 310:927-35; Kaseretal, 2003 Biochem. Biophys Res. Commun., 309:286-90).

Resistin expression in human monocytes was markedly increased by treatment with endotoxin and pro-inflammatory cytokines (Lu et al, 2002 FEBS Lett., 530:158-62; Kaser et al, 2003 cited above). Recombinant resistin up-regulates cytokines and adhesion molecules expression on human endothelial cells (Verma et al, 2003 Circul., 108:736-40; Kawanami et al, 2004 Biochem. Biophys Res. Commun., 314:415-9) suggesting a potential role in atherosclerosis. Recently, several studies have suggested that metabolic abnormalities are associated with polymorphisms in the human resistin gene (Tan et al, 2003, J. Clin. Endocrinol. Metab., 88:1258-1263; Smith et al, 2003 Diabetes 52:1611-1618). However, the relationship of resistin to inflammation, insulin resistance and atherosclerosis in humans remains largely unexplored.

There remains a need in the art for methods and compositions employing resistin in the areas of therapy and diagnosis of disease.

### SUMMARY OF THE INVENTION

In one aspect, the invention provides a diagnostic method for determining the risk or progression of cardiovascular disease in a mammalian subject by employing resistin as a novel biomarker for such diseases. Thus, the method of this invention involves determining the risk or progression of a cardiovascular disease by measuring the level of resistin protein in a biological fluid of a mammalian subject. This measured level is compared to a standard of resistin levels in a population. An elevated resistin level compared to the standard is predictive of increased risk of disease. In one embodiment, a resistin level greater than that of the lowest 25% of the resistin levels forming the population is indicative of risk of cardiovascular disease. In another embodiment, if the subject's resistin level is greater than that of 50% of the resistin levels forming the population, an intermediate risk of cardiovascular disease is diagnosed. In still another embodiment, a resistin level greater than that of 75% of the resistin levels forming the population is indicative of high risk of cardiovascular disease or progression of existing cardiovascular disease. In another embodiment, a resistin level greater than that of 80% of the resistin levels forming the population is indicative of highest risk of disease.

In another aspect, a method of this invention further involves measuring the concentration of a second biomarker of cardiovascular disease or a second inflammatory biomarker in the sample and correlating the resistin level with the level of the second biomarker, wherein the combination of resistin concentration and second biomarker concentration is predictive of cardiovascular risk.

In another aspect, a method of this invention involves repeatedly measuring circulating resistin over time to monitor the progression of cardiovascular disease risk.

In one embodiment of these methods, plasma or serum resistin levels are predictive of risk of cardiovascular disease, such as atherosclerosis in mammalian subjects that are asymptomatic for cardiovascular disease and/or are not diabetic. In a further embodiment, the method of the present invention predicts cardiovascular disease risk for mammalian subjects symptomatic for metabolic syndrome. In still a further embodiment, the method of this invention assesses the risk of cardiovascular disease for subjects with diabetes. In yet another embodiment the method of this invention may be employed to track risk of such disease over time in a subject.

In yet a further aspect, the invention provides a method for treating or retarding the progression of an inflammatory disorder or cardiovascular disease in a mammalian subject by reducing the level or effect of the subject's circulating resistin.

Other aspects and advantages of the present invention are described further in the following detailed description of the preferred embodiments thereof.

### BRIEF DESCRIPTION OF THE DRAWINGS

FIG. 1A is a bar graph showing that human macrophages in cell culture express resistin. Resistin is induced by LPS in a dose-dependent manner during human macrophage differentiation ex vivo. LPS is an endotoxin that causes acute inflammation, and has been previously shown to cause insulin resistance in rodents and humans. Expression of resistin is measured on Days 1, 3 and 7 following isolation and culture of human peripheral blood monocytes under macrophage differentiation conditions. Results are the mean±standard error (SEM) of three separate experiments with triplicate samples. The ANOVA F statistic for change of resistin mRNA expression during differentiation was 7.06 (p<0.01) and the p values for post hoc t-tests are depicted in the Fig. *p<0.01.

FIG. 1B is a bar graph demonstrating that resistin mRNA is induced by endotoxin in a dose-dependent manner in primary human macrophage cultures. The ANOVA F statistic for change of resistin mRNA expression in response to increasing concentration of LPS (24 h treatment) was 423.57 (p<0.001). P values for post hoc t-tests are depicted in the Figure. *p<0.001. For LPS dose response studies, results (mean±(SEM)) of representative experiments, with triplicate samples, are presented. Similar results were obtained in two independent experiments.

FIG. 1C is a bar graph showing that resistin protein secretion by human macrophages is induced by endotoxin LPS in a dose-dependent manner. The ANOVA F statistic for change of resistin protein secretion in response to increasing concentration of LPS (24 h treatment) was 35.36 (p<0.001). P values for post hoc t-tests are depicted in the Fig. *p<0.001. For LPS dose response studies, results (mean±(SEM)) of representative experiments, with triplicate samples, are presented. Similar results were obtained in two independent experiments.

FIG. 2A is a bar graph that demonstrates that the antidiabetic drug rosiglitazone at the indicated concentrations suppresses resistin mRNA in LPS-stimulated macrophages in cell culture.

FIG. 2B is a bar graph that demonstrates that the antidiabetic drug rosiglitazone at the indicated concentrations suppresses resistin protein production in LPS-stimulated macrophages in cell culture.

FIG. 3A is a graph showing that the induction of resistin mRNA by LPS occurs between 6-24 hours after LPS exposure of human macrophages in cell culture.

FIG. 3B is a graph showing that the induction of resistin protein by LPS occurs between 6-24 hours after LPS exposure of human macrophages in cell culture.

FIG. 3C is a graph showing the induction of TNFα, another inflammatory cytokine, whose induction precedes that of resistin after LPS exposure of human macrophages in cell culture. This suggests that TNFα (and perhaps other cytokines) stimulate resistin production.

FIG. 4A further demonstrates that endotoxin induction of resistin occurs after induction of TNFα. Primary cultures of human macrophages were treated with LPS (1 μg/ml) for various times. This bar graph shows the time course of induction of resistin mRNA. The ANOVA F statistic for the change in resistin mRNA over time was 105.45 (p<0.001).

FIG. 4B is a bar graph showing the time course of induction of TNFα mRNA in the same experiment. The ANOVA F statistic was 34.57 (p<0.001)

FIG. 4C is graph showing the time course of secretion of resistin, TNFα, and sTNFR2 into medium. ANOVA F statistics for the effect of LPS on resistin (66.51, p<0.001), sTNR2 (12.86; p<0.001) and TNFα (20.48; p<0.001) were highly significant. Maximal secreted protein levels: resistin, 21.9 ng/ml/mg; TNFα: 207.2 ng/ml/mg; sTNFR2: 39.3 ng/ml/mg. Results of representative experiments with triplicate samples are expressed as mean±(SEM). Similar results were obtained in three independent experiments.

FIG. 5A is a bar graph showing that TNFα, an endotoxin-induced cytokine, regulates and induces resistin mRNA expression from cultured primary human macrophages. The ANOVA F statistic for the effect of increasing TNFα concentrations on resistin was 23.81 (p<0.001). P values for post hoc t-tests, is depicted in the Fig. *p<0.001.

FIG. 5B is a bar graph showing that TNFα induces resistin protein secretion by primary human macrophages. ANOVA F statistic for the effect of TNFα on resistin was 79.85 (p<0.001). The P values for post hoc t-tests are depicted in the Fig. *p<0.005. Results of representative experiments with triplicate samples are expressed as mean±SEM. Similar results were obtained in two independent experiments.

FIG. 5C is a bar graph showing that antibody to TNFα (TNFAB) partially blocks the induction of resistin mRNA by LPS, as does antibody to other cytokines IL-6 (IL-6AB) and IL-6 (IL1AB), but not control antibody (contAB).

FIG. 5D is a bar graph showing that a combination of the antibodies even more effectively blocks induction of resistin RNA by LPS in cultured macrophages. These cytokines (IL-6, IL1 and TNFα) are all increased in obesity and have been linked to insulin resistance.

FIG. 5E is a bar graph showing that LPS (1 μg/ml) induction of resistin is abrogated by antibody neutralization of cytokines TNFα, IL-6 and IL-6, (7.5 μg/ml per antibody). ANOVA F statistic for the effect of neutralizing antibodies on resistin was 3.08 (p<0.05). P values for post hoc t-tests: *p<0.05, **p<0.001 versus IgG. Results are the mean±standard error (SEM) of three separate experiments with triplicate samples. The presence of the antibodies is indicated by the “+” sign under the appropriate column.

FIG. 6A is a graph indicating that LPS dramatically induces resistin (♦) serum/plasma levels in humans. The increase is over 400% and is sustained relative to that of an accepted marker of inflammation, soluble TNF-receptor, sTNFR2 (□), which has been independently linked to diabetes, obesity, insulin resistance and atherosclerotic cardiovascular disease.

FIG. 6B is a similar graph showing that plasma resistin and soluble TNFR2 levels were measured serially in 6 normal volunteers for 24 hours before and after intravenous LPS (3 ng/kg) administration. The repeatedly measured ANOVA F statistic for the effect of LPS on plasma resistin (9.25, p<0.001) and sTNR2 (23.65; p<0.001) was highly significant.

FIG. 6C is another graph showing the mean resistin RNA expression in whole blood cells of normal volunteers (n=2) before and after treatment with LPS (3 ng/kg).

FIG. 7A is a bar graph showing inhibition of resistin induction by anti-inflammatory insulin sensitizers. Down-regulation of resistin mRNA is caused by rosiglitazone. ANOVA F statistic for the effect of rosiglitazone on resistin expression was 62.52 (p<0.001). P value for post hoc t-tests, is depicted in the Fig. *p<0.005 versus control.

FIG. 7B is a bar graph showing down-regulation of resistin protein secretion by human macrophages treated with rosiglitazone. The ANOVA F statistic for the effect of rosiglitazone on resistin protein secretion was 29.44 (p<0.001). P value for post hoc t-tests: *p<0.05, **p<0.001 versus control. Cells were pre-treated with rosiglitazone for 24 h and with LPS (1 μg/ml) and rosiglitazone for an additional 24 hours. Results of representative experiments with triplicate samples are expressed as mean±(SEM). Similar results were obtained in three independent experiments.

FIG. 7C is a bar graph showing down-regulation of resistin gene expression by aspirin. The ANOVA F statistic for the effect of aspirin on resistin expression was 61.33 (p<0.001). P values for post hoc t-test. *p<0.01, **p<0.001, ***p<0.0001 versus no ASA. Cells were pre-treated with aspirin for 2 h and with LPS (1 μg/ml) and aspirin for an additional 24 hours. Results of representative experiments with triplicate samples are expressed as mean±(SEM). Similar results were obtained in two independent experiments.

FIG. 7D is a bar graph showing down-regulation of resistin gene expression by NF-κB inhibitor SN50. *p<0.001 versus control peptide by t-test. Cells were pre-treated with SN50 or control peptide at 100 μg/ml for 2 h, and with LPS (1 μg/ml) and SN50 or control peptide for an additional 24 hours. Results are the mean±(SEM) of two independent experiments performed in triplicate.

FIG. 7E is a bar graph showing the induction of resistin by activation of NF-κB. *p<0.05 versus control virus by t-test. Cells were infected with adenovirus expressing activated IκK or control virus for 24 h. Results of representative experiments with triplicate samples are expressed as mean±(SEM). Similar results were obtained in two independent experiments.

FIG. 7F is a bar graph showing the down regulation of resistin gene expression by inhibitors of p38 and p42 MAP-kinase. The ANOVA F statistic for the effect of the MAP-kinase inhibitor on resistin expression was 11.54 (p<0.005). P value for post hoc t-tests are is depicted in the Fig. *p<0.005 versus control. Cells were pretreated with 50 μM PD98059 or 2.5 μM SB20358 for 2 h and with LPS (1 μg/ml) and PD98059 or SB20358 for an additional 24 hours. Results are the mean±(SEM) of two independent experiments performed in triplicate.

FIG. 8 is a graphical model to explain hyperresistinemia in mice and human obesity despite the species differences in the source of plasma resistin. Circulating inflammatory cytokines TNFα and IL-6 are depicted because of their role in resistin induction in human macrophages and implication in insulin resistance. Other cytokines and inflammatory markers may also contribute to insulin resistance and/or resistin induction.

FIG. 9A is a bar graph showing that coronary artery calcification (CAC) scores increased across plasma resistin quartiles in men (trend p=0.01). Coronary artery calcification (CAC) data is illustrated as the log (CAC+1) for ease of presentation. Median and inter-quartile range (IQR) CAC scores are shown beneath the plot.

FIG. 9B is a bar graph showing that CAC scores increased across plasma resistin quartiles in women (trend p=0.05). CAC data is illustrated as the log (CAC+1) for ease of presentation. Median and inter-quartile range (IQR) CAC scores are shown beneath the plot.

### DETAILED DESCRIPTION OF THE INVENTION

In response to the need in the art, the present invention provides methods for evaluating the risk of a mammalian subject developing a cardiovascular disease or evaluating the progression of a cardiovascular disease by employing resistin as a biomarker or screening tool. The present invention provides a correlation between serum resistin levels or plasma resistin levels in mammalian subjects and risk level for the development or progression of cardiovascular disease.

As evidenced in the examples below and summarized here, the inventors found that plasma levels of resistin were also associated with inflammatory markers in a large, non-diabetic sample of human subjects as well as in a sample of human type 2 diabetic subjects. Resistin was associated with coronary artery calcification (CAC), a measure of coronary atherosclerosis, even after controlling for established risk factors, metabolic syndrome and CRP levels. Additionally the examples demonstrate that inflammatory endotoxin induces resistin in primary human macrophages via a cascade involving the secretion of inflammatory cytokines that circulate at increased levels in obesity. This is attenuated by drugs with dual insulin sensitizing and anti-inflammatory properties that converge on NF-κB. In human subjects, experimental endotoxemia, which produces an insulin resistant state, causes a dramatic rise in circulating resistin levels. Moreover, in type 2 diabetics, serum resistin levels are correlated with levels of soluble TNFα receptor, an inflammatory marker linked to obesity, insulin resistance, and atherosclerosis.

A. Diagnostic/Prognostic Methods for Evaluating Risk of Cardiovascular Disease

The present invention provides novel methods for evaluating the risk of cardiovascular diseases or coronary artery diseases by evaluating levels of resistin in a mammalian subject, preferably a human. As used herein, the term “resistin” may be defined as described in International Patent Publication No. WO 00/64920, incorporated herein by reference, and by the nucleotide and amino acid sequences set out therein. Resistin sequences have also been described in other publications and identified as FIZZ3 (see other publications cited herein).

As used herein the terms cardiovascular disease (CVD) and coronary artery disease (CAD) are intended to encompass, but are not limited to, heart disease, atherosclerosis, microvascular disease, hypertension, stroke, diabetic angiopathies, myocardial infarction, acute coronary syndrome, unstable angina, and diabetic retinopathy.

As one embodiment of this invention, a method for diagnosing risk of CVD or CAD involves measuring the resistin levels in a biological sample from a mammalian subject. As used herein, the term “biological sample” includes, without limitation, any sample from a human patient, e.g., a body fluid, such as blood, serum or plasma, but also possibly urine, saliva, and other fluids or tissue. Preferably the biological sample is a blood sample, such as a serum or plasma sample.

The measurement of the concentration of resistin protein in the biological sample may employ any suitable resistin antibody to detect the protein. Such antibodies may be presently extant in the art or presently used commercially, or may be developed by techniques now common in the field of immunology. As used herein, the term “antibody” refers to an intact immunoglobulin having two light and two heavy chains or any fragments thereof. Thus a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, a light chain variable or complementarity determining region (CDR) sequence, etc. A recombinant molecule bearing the binding portion of an anti-resistin antibody, e.g., carrying one or more variable chain CDR sequences that bind resistin, may also be used in a diagnostic assay of this invention. As used herein, the term “antibody” may also refer, where appropriate, to a mixture of different antibodies or antibody fragments that bind to resistin. Such different antibodies may bind to a different portion of the resistin protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds resistin or a particular sequence of resistin as defined above or described in International Patent Publication No. WO/0064920 may be employed in the methods of the present invention, regardless of how the antibody or mixture of antibodies was generated.

Similarly, the antibodies may be tagged or labeled with reagents capable of providing a detectable signal, depending upon the assay format Such labels are capable, alone or in concert with other compositions or compounds, of providing a detectable signal. Where more than one antibody is employed in a diagnostic method, the labels are desirably interactive to produce a detectable signal. Most desirably, the label is detectable visually, e.g. calorimetrically. A variety of enzyme systems operate to reveal a calorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting resistin-antibody complex in applicable assays. Still other labels include fluorescent compounds, radioactive compounds or elements. Preferably, an anti-resistin antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The anti-resistin antibodies or fragment useful in this invention are not limited by the particular detectable label or label system employed. Thus, selection and/or generation of suitable anti-resistin antibodies with optional labels for use in this invention is within the skill of the art, provided with this specification, the documents incorporated herein, and the conventional teachings of immunology.

Similarly the particular assay format used to measure the resistin in a biological sample may be selected from among a wide range of immunoassays, such as enzyme-linked immunoassays, such as those described in the examples below, sandwich immunoassays, homogeneous assays, or other assay conventional assay formats. One of skill in the art may readily select from any number of conventional immunoassay formats to perform this invention.

Other reagents for the detection of protein in biological samples, such as peptide mimetics, synthetic chemical compounds capable of detecting resistin may be used in other assay formats for the quantitative detection of resistin protein in biological samples, such as Western blots, flow cytometry, etc.

The measurement of resistin, preferably in plasma or serum, serves as a biomarker for CVD risk. According to the method of this invention, to determine the risk or progression of a cardiovascular disease, the level of resistin protein in a biological fluid of a mammalian subject is measured and compared to a reference standard of resistin levels in a population. An elevated resistin level compared to said standard is predictive of increased risk of disease.

The reference standard is that established by measuring resistin values of a normal population sample, which is naturally composed of mammalian subjects of varying degrees of cardiovascular health, from healthy, through various increasing risks of CVD/CAD to those suffering from CVD/CAD. Thus, the standard is preferably provided by using the same assay technique as is used for measurement of the subject's resistin levels, to avoid any error in standardization. As demonstrated in the examples below, the relative level of risk of CVD can be determined based upon the increase of resistin as compared against the resistin levels of a population. As demonstrated by the examples below, there is almost a linear increased CVD/CAD risk with increased levels of serum/plasma resistin.

Thus, in one embodiment, where the subject's resistin level is within the lowest 25% of the resistin levels forming the population, the subject has a “normal” level of resistin, or a low risk of cardiovascular disease. Thus, where the subject's resistin level is greater than that of the lowest 25% of the resistin levels of the population, this measurement is indicative of some risk of cardiovascular disease. For example, in another embodiment, where the subject's resistin level is within the lowest 25-50% of the resistin levels forming the population, the subject has a low intermediate, but increased risk of cardiovascular disease. In circumstances in which the subject's resistin level is greater than that of 50% of the resistin levels forming the population, the subject is diagnosed as having an intermediate risk of cardiovascular disease. Similarly, where the subject's resistin level is greater than that of 75% of the resistin levels forming the population, the subject has a high risk of developing cardiovascular disease or is evidencing progression of existing cardiovascular disease. Finally, where the subject's resistin level is greater than that of 80% the resistin levels of the standard population, the subject is demonstrating the highest risk of disease and/or progressive cardiovascular disease.

As described above, “normal” levels of resistin in a population, i.e., the levels in the lowest 25% of the standard population, can vary based upon any variables in an individual assay used for measurement and the standardization of regents employed in such assay. Therefore, in one embodiment of this invention, i.e., that based upon the assay and antibody employed in the examples below, the lowest 25% of the population evidenced a “normal” level of resistin as falling below about 4 ng/ml. However, in another assay, the “normal” value may be below 3 ng/ml or below 15 ng/ml. Increasingly sensitive assays may further lower the “normal” or lowest range of resistin in a population. For example, according to the ELISA assay employed in the examples below, levels of serum/plasma resistin falling within a measurement of about 1.5 to about 4 ng/ml are indicative of “normal” or relatively low risk of CVD. The specific range detected in the examples for the “low risk” designation was 1.66 ng to 4.13 ng resistin per ml of sample.

According to this invention, a level of serum/plasma resistin falling within a measurement of resistin values of the lowest 25 to 50% of the population is indicative of low intermediate, but increasing, risk of CVD/CAD. In one embodiment of this invention exemplified by the assay below, such a low intermediate risk value is above about 4 to about 5.5 ng/ml resistin. The specific range detected in the examples for the “low, intermediate risk”, i.e., risk of the lowest 25-50% of the standard population was 4.13 ng to 5.46 ng resistin per ml of sample.

Further according to this invention a level of serum/plasma resistin falling within resistin values for the quartile of population falling between 50-75% is indicative of high intermediate, increasing risk of CVD/CAD. In the embodiment of the assay exemplified below, a serum resistin measurement at this quartile was between about 5.5 to about 7.2 ng/ml. The specific range detected in the examples for the “high intermediate risk” designation was 5.46 ng to 7.28 ng resistin per ml of sample.

Finally, according to this invention a measurement of the subject's resistin protein in a biological sample that is greater than about 75% or 80% of the standard population is indicative of high risk of developing CVD or the presence of existing, progressive CVD or CAD, particularly atherosclerosis. In the embodiment of the assay exemplified below, such a high risk profile is demonstrated by a serum resistin value of 7.3 ng/ml or greater. In another embodiment the high risk profile is identified by a serum resistin value of about 10 ng/ml, 15 mg/ml or greater. Still other values may be determined relative to the population quartiles applicable to the particular assay.

Of note are the resistin levels demonstrated by the endotoxin induction levels found in the Examples 6-12 below. These examples show a putative maximum increase in resistin due to inflammation of 18-19 ng/ml or more, or an increase of 3-5 fold, with some patients being up to triple that number. Thus, dependent upon the determination of “normal” value for any particular assay in the standard population, each increase in resistin levels for each 25% of the standard population is diagnostic of an additional level of risk for the development or progression of CVD/CAD.

As demonstrated below in the examples, such levels of resistin in plasma or serum demonstrate a reliable assay for CVD/CAD risk detection when measured in human subjects that are asymptomatic for heart disease and non-diabetic. Stronger correlation is demonstrated for human subjects with type 2 diabetes and/or metabolic syndrome or Syndrome X. It is anticipated that even greater correlation in patients with some other symptoms of CVD/CAD can be shown, thereby establishing resistin as a novel and useful biomarker for both risk of CVD and CAD in otherwise healthy patients with no symptoms and as a biomarker to monitor the CV status of patients with existing CVD/CAD disease.

Such evaluation of resistin levels independent of a second biomarker of CVD/CAD provides useful indicators of relative risk of CVD/CAD or progression of existing disease.

In still a further embodiment of methods according to this invention, a risk evaluation of CVD or CAD may be performed by measuring resistin levels in combination with measuring one or more second or other CVD/CAD biomarker. Such second or additional biomarkers include, without limitation, coronary artery calcification, high-sensitivity C-reactive protein, markers of inflammation, lipoprotein (a), homocysteine, markers of fibrinolytic and hemostatic function, such as fibrinogen, D-dimer, tPA, plasminogen activator inhibitor 1 antigen, inflammatory markers, such as TNFα, LpPLA2, BNP, IL-18, IL-14, IL-6, TNF-α, solTNFR1 and CD40L, among others, as well as measurements of HDL, LDL and other tradition risk factors for CVD, such as those listed in Bassuk et al, cited above. Correlation between the resistin level and a level indicative of CVD risk for the known second biomarker further confirms the risk or progression of CVD. Thus the measurement of resistin may serve to confirm indications of CVD provided by assays for known biomarkers. Alternatively the measurement of resistin may serve to more accurately diagnose the CVD/CAD risk than the known biomarkers, such as CRP.

In yet a further embodiment, the method of this invention can include the step of repeatedly measuring resistin levels over a given time period, and thereby serve to monitor the progress of patients with CVD. The method may be useful to determine the degree of success of a particular therapeutic regimen for CVD/CAD and may indicate circumstances in which a change of therapy is necessary.

B. Therapeutic Methods for Treating Cardiovascular Disease or Inflammation

As a corollary to the inventors' determination that resistin levels are a biomarker for CAD/CAV, the present invention further provides novel therapeutic treatments for retarding the progression of CVD/CAD and/or an inflammatory disorder. Such inflammatory disorders, include without limitation, diabetes, obesity, insulin resistance, and diseases that arise from atherosclerotic cardiovascular disease, such as stroke, kidney failure, blindness and embolism, among others. Such a method provides a therapeutic regimen comprising administering to a patient an amount of a resistin antagonist that is sufficient to reduce circulating resistin. Since the CVD/CAD risk levels of resistin increase linearly with increases of resistin in plasma or serum over the standard population, described above, this method seeks to reduce resistin levels to successively lower risk level values. For example, for patients in the very high risk category based on serum resistin levels, i.e., the values in the top 75% of the standard population, the method involves neutralizing serum resistin to a concentration falling within the next lowest level of the standard. However, as with cholesterol levels, it is considered desirable to reduce high resistin levels by any value under that of the starting high risk level of resistin. Treatment is repeated so that resistin levels are progressively reduced by increments until the resistin level is stabilized in the lowest percentile of the standard population as possible, i.e., as low or as close to normal/low risk/first 25% of the standard population as possible for the particular patient.

Thus in one embodiment, the method of the invention is directed to treating or retarding the progress of an inflammatory disorder or a cardiovascular disorder in a mammalian subject by reducing the level or effect of the subject's circulating resistin. Desirably, the levels are reduced by at least 10% of presenting levels. Still more desirably, the levels are reduced by at least 20% of presenting levels. Using the assay described above, one may measure the subject's resistin levels by comparing the subject's level to the resistin levels in a standard population. Thus, it is also desirable to reduce the subject's level to a level less than those with the highest quartile of the population of said standard, i.e., a 75% cut-point. According to this method, treatment may be continued to reduce the subject's resistin level to a level within or less than the 50-75% quartile of the standard population. According to another embodiment of this invention, the method is employed to reduce the subject's resistin level to a level less than that of the top 50% of the standard population. Of course, practice of the method is most desirable, where it reduces the subject's level to a level within that of the lowest 25% of the standard population.

For example, according to the examples and using an assay to measure resistin as described in the examples herein, the method involves treating the patient to neutralize resistin levels to values of about 10 ng resistin/ml in a suitable biological fluid. Preferably the biological fluid is plasma or serum. In yet another embodiment, the method is performed to reduce the amount of circulating resistin to less than 40% of normal values. For example, according to the examples and using an assay to measure resistin as described in the examples herein, the method involves treating the patient to neutralize resistin levels to less than 7.2 ng/ml. In yet another embodiment, the method is performed to reduce the amount of circulating resistin to less than 20% of normal values. For example, according to the examples and using an assay to measure resistin as described in the examples herein, the method involves treating the patient to neutralize resistin levels to less than 5.5 ng/ml. Still a further embodiment of this invention involves administering to a patient an amount or course of a resistin antagonist to reduce the circulating resistin level to approximately normal values of resistin. According to the examples and using an assay to measure resistin as described in the examples herein, the method involves treating the patient to neutralize resistin levels to less than about 4 ng/ml. As mentioned above, the specifically defined concentrations depend upon the exemplified assay described herein. However, other standard populations are developed for use with other assays, and the concentrations are expected to vary.

These methods may involve repeatedly administering the antagonist or providing the patient with a course of therapy in which the circulating resistin level is maintained at a desired threshold level, as described herein.

Such therapeutic methods are useful for patients having existing CVD/CAD, for patients having metabolic syndrome, for diabetic patients, for patients having an inflammatory disorder, such as diabetes or general hyperresistinemia, or for asymptomatic patients having a circulating resistin level of greater than normal values of circulating resistin.

The term “resistin antagonist” is meant any compound that can reduce circulating resistin to the above noted lower risk levels upon treatment than is presented before treatment. In one embodiment, the antagonist prevents the binding of resistin to its naturally occurring receptor. Thus, such a compound may be a synthetic drug, an anti-resistin antibody or fragment thereof, or a therapeutic composition that decreases expression of resistin. Such resistin antagonists useful in this therapeutic method may be known compounds available commercially or in the prior art.

According to this method, suitable amounts and formulations of the selected resistin antagonist for administration to a patient, preferably a human patient, to accomplish the desired reduction in circulating resistin may be chosen by an attending physician depending upon relative factors. For example, dosages of the resistin-reducing compounds selected vary with the particular compositions employed (the nature of the antagonist, e.g., proteinaceous, synthetic chemical, etc.), the half-life of the compound, the identity and/or stage of the cardiovascular disease or inflammatory disease, the presenting resistin level of the patient, the patient's age, weight, sex, general physical condition, the route of administration, any other medications and treatment, as well as the subject's medical history. Precise dosages can be determined by the administering physician based on experience with the individual subject treated. An effective therapeutic dosage contains an amount sufficient to reduce circulating resistin levels, and preferably sufficient to reduce starting resistin levels by about 20% or more.

Similarly, the routes of administration, dosage regimen and dosage frequency depends upon the factors identified above and upon the response of the patient to the therapy, as determined by periodic evaluation of the resistin level.

C. Data Supporting Methods of the Invention

The inventors have discovered that resistin levels are associated with a cardiovascular disease state, such as coronary atherosclerosis, even after controlling for established risk factors, metabolic syndrome, and plasma CRP levels. The following examples establish the relationship of circulating resistin with diverse inflammatory markers, as well as with coronary atherosclerosis. Further the examples demonstrate that resistin levels are predictive of a CVD, e.g., coronary atherosclerosis, in humans, independent of CRP.

Resistin represents a unique, species specific link between metabolic signals, inflammation and atherosclerosis, particularly in humans. The inventors found that plasma resistin levels were associated with markers of inflammation, but not insulin resistance, in both SIRCA, a study of asymptomatic non-diabetic subjects, and in a type 2 diabetic sample. Further, resistin levels were found to be significantly associated with coronary atherosclerosis in SIRCA even after controlling for multiple established risk factors and the presence of metabolic syndrome. In fact, plasma levels of resistin, unlike CRP, provided incremental value in the association with coronary artery calcification (CAC) in subjects with the metabolic syndrome.

Also provided below is evidence that acute endotoxemia dramatically (>7-fold) elevates plasma levels of resistin in humans. Consistent with recent small clinical studies (Vendrell et al, 2004 Obes. Res., 12:962-71; Shetty et al, 2004 Diabetes Care. 27:2450-7), these findings suggest that, in contrast to other adipokines, resistin expression and secretion in humans may be regulated by innate inflammatory signals. Endotoxemia is known to produce a state of insulin resistance in humans (Agwunobi et al, 2000 J. Clin. Endocrinol. Metab., 85:3770-8).

In SIRCA, plasma resistin levels were strongly and independently correlated with sol TNF-R2, an index of TNFα system activation (Bemelmans et al, 1996 Crit. Rev. Immunol., 16: 1-11), and IL-6. Both TNFα and IL-6 are derived from adipose tissue as well as macrophages and increased levels of these inflammatory cytokines have been linked to obesity, insulin resistance and atherosclerotic CVD (Moller, 2000 Trends Endocrinol. Metab., 11:212-7). The inventors found that resistin levels also correlated significantly with sol ICAM-1 and LpPLA2, plasma markers thought to derive from monocytes and the endothelium rather than adipose tissue. However, resistin levels were not associated with plasma levels of CRP, which is largely secreted by the liver. The contribution of innate inflammatory cells to the circulating resistin levels, versus that of adipocytes, is greater in the relatively lean, non-diabetic population than in other studies that have focused on resistin levels in obesity or type 2 diabetes (see the references cited above or cited in Reilly et al, 2005 Circulation, 111:932-939, incorporated herein by reference).

Therefore, resistin levels in SIRCA subgroups and in type 2 diabetic samples were examined. Although, these studies were recruited separately and were not designed to compare levels across study samples, the findings are consistent with modest increases in resistin in overweight and type 2 diabetic samples as has been published in other studies of obesity. Obesity and type 2 diabetes are associated with activation of innate immune pathways and chronic inflammation (Haffner, 2003, cited above). The consistent correlation of resistin with sol TNF-R2 in both SIRCA and diabetic subjects, and the increase in circulating resistin during endotoxemia in healthy humans, strongly define resistin as an inflammatory adipokine across a variety of settings in humans and suggest distinct but overlapping sources and functions for innate inflammatory signals in human pathophysiology (Rader, 2000 N Engl. J. Med., 343:1179-82). The finding of stable resistin levels in healthy subjects over a 24-hour period in the GCRC suggest also that measurement of plasma levels of resistin in cross-sectional studies is useful.

Plasma resistin levels were significantly associated with CAC in the SIRCA sample. Although not a direct measure of coronary atherosclerosis, autopsy studies have shown that CAC is a quantitative measure of coronary atherosclerosis (Rumberger et al, 1994 Am. J. Cardiol., 73:1169-73) and recent studies support its utility as a predictor of CVD events in asymptomatic samples, even at relatively low scores (Kondos et al 2003 Circul., 107:2471-6; Pletcher et al, 2004 Arch. Intern., Med., 164:1285-92). The association of resistin with CAC was maintained even after controlling for established risk factors, as well as the presence of the metabolic syndrome and plasma levels of CRP. Because the metabolic syndrome is a strong risk factor for atherosclerotic CVD but the optimal definition for use in practice remains unclear, additional biomarkers are being sought to refine CVD risk prediction. CRP is promising in this regard (Ridker et al, 2003 Circul. 107:391-7; Sattar et al, 2003 Circul., 108:414-9). When plasma resistin was compared to CRP in association with CAC in metabolic syndrome subgroups, notably, in metabolic syndrome subjects, resistin levels further predicted increased CAC whereas CRP levels did not. These clinical correlations are consistent with recent reports showing that recombinant resistin induced cytokine, chemokine and adhesion molecule expression in human endothelial cells (Verma, 2003 and Kawanami, 2004, both cited above), whereas adiponectin opposed the effect of resistin on adhesion molecules (Kawanami, 2004, cited above). Plasma resistin thus is useful as a biomarker in the diagnosis and tracking of CV risk prediction beyond methods available in the prior art.

In the examples below, the inventors also demonstrate that the endotoxin lipopolysaccharide (LPS), a potent inflammatory stimulant, dramatically increases resistin production by inducing secretion of inflammatory cytokines such as TNFα. This is blocked both by aspirin and rosiglitazone, drugs that have dual anti-inflammatory and insulin sensitizing actions and have been shown to antagonize NF-κB Indeed, activation of NF-κB is sufficient to induce resistin expression, and loss of NF-κB function abolishes LPS induction of resistin. Resistin serum levels are increased dramatically by endotoxemia in human subjects, and correlate with a marker of inflammation in patients with type 2 diabetes. Thus, systemic inflammation leads to increased resistin production and circulating levels in humans. The increased level of resistin in humans with obesity is likely an indirect result of elevated levels of inflammatory cytokines characteristic of states of increased adiposity. Hence, obesity and acute inflammation are both hyperresistinemic states associated with insulin resistance.

### D. EXAMPLES

The following examples illustrate various aspects of this invention. Examples 1 through 5 demonstrate that plasma resistin levels were associated with markers of inflammation, body fat, insulin resistance, and inflammatory markers. More particularly, the examples demonstrate whether levels of resistin are associated with coronary atherosclerosis, as measured by coronary artery calcification (CAC) at electron beam tomography (EBT), a quantitative index of atherosclerosis, in 879 to 896 asymptomatic subjects in the Study of Inherited Risk of Coronary Atherosclerosis (SIRCA). Resistin levels, particularly plasma resistin levels, were positively associated and correlated significantly with levels of inflammatory markers including soluble TNFα-Receptor-2 (p<0.001), interleukin-6 (p=0.04 in later studies) and LpPLA2 (p=0.002 in later studies), but not measures of adiposity or insulin resistance in multivariable analysis. In the preliminary multivariable analyses, female gender (p<0.001), TNFα-R2 (P<0.001), IL-6 (P=0.13) and LpPLA2 (p=0.003 were positively associated and HDL cholesterol (p=0.05) and alcohol intake (0.04) were inversely associated with log transformed plasma resistin levels. Resistin levels also were associated (odds ratio and 95% confidence interval in ordinal regression) with increasing CAC after adjusting for age, gender and established risk factors [OR 1.23 (1.03 to 1.52), p=0.03] and controlling further for NCEP defined metabolic syndrome and plasma C reactive protein (CRP) levels [OR 1.25 (1.04 to 1.50), p=0.01]. In fact, addition of resistin levels to CRP significantly improved the association with CAC (p=0.05), but addition of CRP levels to resistin did not (p=0.05). In subjects with metabolic syndrome, resistin levels further predicted CAC, whereas CRP levels did not.

Examples 6 through 12 show the effect of endotoxin and cytokines on resistin gene and protein expression in human primary blood monocytes differentiated into macrophages and in normal human subjects. This data demonstrates that, in human macrophages, an inflammatory cascade with secretion of cytokines, including TNFα and IL-6, is sufficient and necessary for the induction of resistin. Insulin sensitizers that have anti-inflammatory properties, including a synthetic PPARγ agonist as well as aspirin, both suppress macrophage resistin expression, as does direct inhibition of NF-κB. Experimental endotoxemia in healthy volunteers, a well established model of gram negative bacterial inflammatory response in humans (see, e.g., Martich et al 1993 Immunobiol., 187:403-416), induces a dramatic elevation of circulating resistin levels. Hence, resistin gene and protein expression are increased by inflammatory stimuli both ex vivo and in vivo. Inflammation is a hyperresistinemic state in humans, and cytokine induction of resistin contributes to insulin resistance in endotoxemia, obesity, and other inflammatory states.

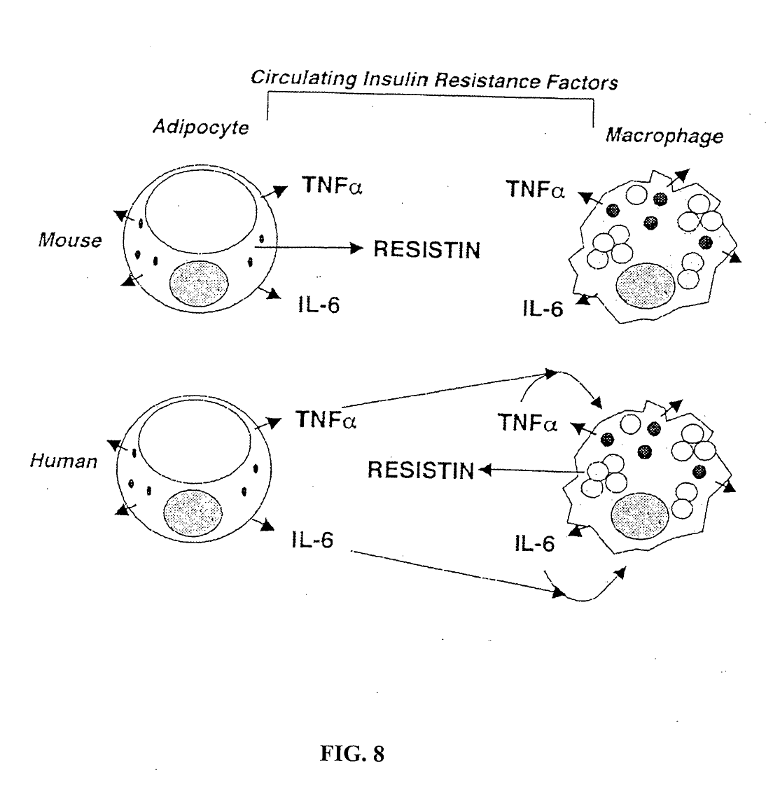

The examples provide evidence that, whereas hyperresistinemia derives directly from adipocytes in obese rodents, human resistin is indirectly regulated by the inflammatory internal milieu of obesity (FIG. 8). Indeed, obesity is associated with elevated levels of cytokines whose systemic administration leads to impaired glucose homeostasis, such as TNFα and IL-6, which are shown to mediate the inflammatory induction of human resistin. Thus, in both species, adipose tissue is an endocrine organ containing adipocytes as well as macrophages that regulates energy metabolism and glucose homeostasis through secretion of multiple factors, including inflammatory cytokines.

Intriguingly, the inventors found a strong correlation between plasma levels of resistin and sTNFR2, the soluble cleavage product of the activated TNFα receptor, in diabetic subjects, comparable to the correlation between resistin and sTNFR2 in the non-diabetic individuals, in whom resistin levels independently correlated with coronary atherosclerotic disease.

LPS binds to pathogen associated molecular pattern (PAMP) innate immune receptors, such as CD14 and Toll like Receptor 4 (TLR4), activating signal cascades involving NF-□B and MAP-Kinase and thereby inducing the transcription and secretion of early cytokines including TNFα and IL-6. The following examples show that these early cytokines are responsible for secondary induction or enhancement of resistin expression in macrophages. Hyperresistinemia impairs glucose homeostasis in rodents, and inflammatory states are associated with insulin resistance, which may serve as a physiological attempt to increase the provision of glucose to the brain under stress conditions. Indeed, induction of acute inflammation by administration of LPS causes insulin resistance in humans. The examples below demonstrate the concomitant induction of resistin. Interestingly, the peak in TNFα and IL-6 levels after LPS administration to humans precedes a phase of prolonged insulin resistance that begins ˜6 h post LPS administration, closely approximating the time course of resistin induction. Hence resistin is a potential mediator of insulin resistance in humans with acute inflammation. Moreover, obesity is associated with activation of innate immunity, including the inflammatory mediators that induce resistin. In this context it is intriguing that resistin levels are increased in obesity, and that insulin sensitizing agents such as aspirin and rosiglitazone, with disparate primary molecular targets, antagonize resistin induction. Indeed, TZD suppression of resistin levels has recently been correlated with hepatic insulin sensitization.

These examples do not limit the scope of this invention which is defined by the appended claims. One skilled in the art will appreciate that although specific reagents and conditions are outlined in the following examples, modifications can be made which are intended to be encompassed by the spirit and scope of the invention.

### Example 1

### Protocols for Determining that Resistin is an Independent Inflammatory Marker of Atherosclerosis

Three experiments were performed on study subjects as described below. The University of Pennsylvania Institutional Review Board approved all three study protocols. Informed consent was given by all subjects.

A. Asymptomatic Patients

In one experiment, plasma levels of resistin were examined for association with inflammatory markers, metabolic parameters and coronary artery calcification (CAC), a measure of coronary atherosclerosis, in 879 asymptomatic, non-diabetic subjects in the Study of Inherited Risk of Coronary Atherosclerosis (SIRCA). Test subjects were enrolled into SIRCA, a cross-sectional study of factors associated with CAC in a community based sample of asymptomatic subjects and their families. Study design and initial findings are as described in Reilly et al, 2003a Arterioscler. Thromb. Vasc. Biol., 23:1851-56; Reilly et al, 2004a Circul., 110:803-809; Reilly et al, 2004b Atherosclerosis, 173:69-73, all incorporated herein by reference). Subjects were included if they were healthy men aged 30-65 or women aged 35-70 who had a family history of premature coronary artery disease (CAD) (before age of 60 in male and age 70 in female first degree relative). Exclusions included evidence of clinical CAD (myocardial infarction, coronary revascularization, angiographic evidence of CAD, or ischemia at cardiac stress test) and serum creatinine >3.0 mg/dl. This experiment used on unrelated non-diabetic subjects recruited to SIRCA (n=879).

B. Type 2 Diabetic Subjects

Plasma resistin levels were measured, during the same time period as for SIRCA, in subjects of an additional clinical research study (Reilly et al, 2004c J. Clin. Endocrinol. Metab., 89:3872-8; Lehrke, 2004 PLoS Medic., 1(2):161-168, both incorporated herein by reference). In this experiment, resistin levels were compared to inflammatory markers in a type 2 diabetic sample (n=215). Plasma resistin was measured in a cross-sectional study of CV risk factors in asymptomatic type 2 diabetic subjects (n=215; 167 male and 48 female; 59% Caucasian and 35% African American) recruited through the Diabetic clinics of the University of Pennsylvania Medical Center and the Veterans Affairs Medical Center, Philadelphia, Pa. Further characteristics of the study sample are provided in Tables 1A-1B and in Reilly et al, 2004c, cited above).

C. Healthy Subjects

In another experiment, short term variation in plasma levels was examined by repeated sampling in young healthy control subjects of an additional clinical research study (Reilly et al, 2004c, and Lehrke, 2004, both cited above). Baseline variability in plasma resistin was assessed over a 24 hour period, in healthy young volunteers (n=6; three male and three females; age 24-34; BMI 24.3±1.07) without any past medical history and on no medications. These subjects were recruited to a 60-hour inpatient, General Clinical Research Center (GCRC) protocol designed to assess the metabolic responses to an inflammatory stimulus. Plasma resistin levels were determined in serial blood samples, collected at eight time points over 24 hours prior to the intravenous administration of human-research-grade endotoxin (3 ng/kg) as described in more detail in Reilly et al, 2004c, cited above.

D. Evaluated Parameters.

SIRCA and diabetic study subjects were evaluated at the GCRC at the University of Pennsylvania Medical Center after a 12-hour overnight fast. Study procedures including questionnaire, physical exam, electrocardiogram and blood collection were performed as described (Reilly et al, 2003a; Reilly et al, 2004a; Reilly et al, 2004b, all cited above). Plasma total and HDL cholesterol, triglyceride and glucose levels were measured enzymatically on a Cobas™ Fara™ II (Roche Diagnostic Systems Inc., N.J., USA) in a Center for Disease Control-certified lipoprotein laboratory. LDL cholesterol was calculated using the Friedewald formula. Young healthy participants in the endotoxin protocol had eight blood draws (at 6 am, 8 am, 12 noon, 2 pm, 6 pm, 10 pm, 2 am, and 6 am) during 24 hours of constant routine in the GCRC prior to endotoxin administration.

Plasma resistin levels were measured by enzyme immunoassay (Linco Research, St Charles, Mo.) as described in Osawa, 2004 Am. J. Hum. Genet., 75:678-86. Monoclonal antibodies, raised against recombinant full length Flag-tagged resistin protein, were generated by the inventor, Mitchell Lazar and made available to Linco through the University of Pennsylvania. This antibody does not react with human RELMβ, the other member of this gene family found in humans. Average correlation coefficient for standards was 0.99. The average intra-assay coefficient of variation (c.v.) was 4.6% for low and 1.7% for high resistin standards and 4.3% for fresh aliquots of pooled human plasma, included in duplicate on all plates. Results for plasma samples across different assay plates, for SIRCA, diabetic and healthy young controls, were standardized using ratio of individual plate pooled plasma to the average pooled plasma value for all plates combined. A direct comparison of the Linco assay with kit with another commercially available resistin ELISA (Biovendor) yielded high correlation (R=0.99, p<0.00).

Plasma levels of interleukin-6 (IL-6), soluble TNF receptor 2 (sol TNF-R2) and soluble intercellular adhesion molecule-1 (sol ICAM-1) were measured using commercially available enzyme-immunoassays (ELISAs) according to the manufacturer's guidelines (R+D Systems, Minneapolis). The intra- and inter-assay c.v.'s for pooled human plasma were 8.7% and 10.9% for IL-6, 5.3% and 12.1% for sol TNF R2, and 1.4% and 10.4% for sol ICAM-1. Plasma C reactive protein (CRP) levels were assayed using an ultra high-sensitivity latex turbidimetric immunoassay (Wako Ltd., Osaka Japan) as described (Reilly et al, 2003a, cited above). Plasma levels of lipoprotein-associated phospholipase A₂ (Lp-PLA₂) were measured using a commercial ELISA (PLAC test; diaDexus, SanFrancisco, Calif.). Intra- and inter-assay c.v.'s for pooled plasma were 6.6% and 8.9%. Plasma insulin levels were measured by ELISA (Linco Research, St Charles Mo.). The intra- and inter-assay c.v.'s were 2.9% and 11.6% for pooled human plasma.

Subjects were classified as having the metabolic syndrome using the National Cholesterol Education Program (NCEP) criteria (Executive Summary 2001 JAMA, 285:2486-97) as previously described in the SIRCA sample (Reilly, 2004a, cited above). The homeostasis model (HOMA index=fasting glucose (mmol/L)×fasting insulin (μU/mL)/22.5) (Matthews et al, 1985 Diabetologia, 28:412-9) was employed as a measure of insulin sensitivity. Global CAC scores were determined using customized software (Imatron, San Francisco, Calif.) according to the method of Agatston et al, 1990 J. Am. Coll. Cardiol., 15:827-32 from forty continuous 3-mm thick computed tomograms collected on an EBT scanner (Imatron, San Francisco, Calif.).

E. Statistical Analysis.

Data are reported as median and inter-quartile range (IQR), or mean±SD, for continuous variables, and as proportions for categorical variables. Spearman correlations of plasma resistin levels with other continuous variables are presented. The association of resistin levels with categorical variables was examined using Kruskal-Wallis rank test and Wilcoxon test for trend. Multivariable linear regression modeling was used to identify factors associated log transformed resistin levels (In-resistin). Gender interaction with other variables in the association with plasma resistin levels was assessed using the likelihood-ratio (LR) test. In order to explore the range of resistin values in different human samples, plasma levels were examined in (1) SIRCA subgroups; (a) subjects with BMI >35 (n=72) and (b) subjects with NCEP-defined metabolic syndrome (n=249), (2) type 2 diabetic subjects and (3) young healthy subjects with repeated blood sampling. Change in plasma resistin levels in young healthy subjects was analyzed by repeated measures analysis of variance (ANOVA).

Median CAC scores were compared across plasma resistin quartiles (1.66 to <4.13, 4.13 to <5.46, 5.46 to <7.28, and >7.28 ng/ml) using Wilcoxon test for trend. Ordinal logistic regression is a method appropriate for the analysis of CAC data which has a markedly non-normal distribution and a significant proportion of subjects with no detectable CAC (Reilly et al, 2003a; and Reilly et al, 2004b, cited above). CAC scores were divided into four ordered outcome categories (0, 1-10, 11-100, >100) using published criteria used to approximate no, mild, and moderate coronary atherosclerosis (Rumberger et al, 1999 May Clin. Proc., 74:243-52).

The association of plasma resistin with CAC was assessed in regression models that included: 1) resistin, gender and age, 2) resistin, established risk factors, gender and age, 3) resistin, metabolic syndrome, non metabolic syndrome factors, gender and age, 4) resistin, plasma CRP levels, metabolic syndrome, non metabolic syndrome factors, gender and age. Established risk factors included total (or LDL) and HDL cholesterol, plasma glucose, systolic blood pressure, smoking (current versus never and ex smokers), race, exercise (none versus any), alcohol intake (drinks per week), and use of medications (aspirin, statins, angiotensin converting enzyme inhibitors, and hormone replacement therapy (HRT) in women). In models that contained metabolic syndrome, non metabolic syndrome factors were smoking, exercise, alcohol intake, race, LDL cholesterol, use of medications. Recently, CRP levels were shown to predict CVD in subjects with the metabolic syndrome (Ridker et al, 2003 and; Sattar et al, 2003, both cited above). Because additional biomarkers are being sought to refine CVD risk prediction in the metabolic syndrome, plasma resistin was compared to CRP in their association with CAC in metabolic syndrome subgroups.

The interaction between sex and plasma resistin levels in the association with CAC was assessed in adjusted models using the likelihood-ratio (LR) test. The LR test also was applied to nested models to determine if addition of resistin to CRP levels, or CRP to resistin levels, improved the prediction of CAC. The results of ordinal logistic regression are presented as the odds ratio (OR) of being in higher CAC category for a 5 ng/ml increase in plasma resistin. The proportional odds assumption of ordinal regression, assessed by the Brant test, was satisfied for resistin in all models. Statistical analyses were performed using Stata™ 8.0 software (Stata Corp, College Station, Tex.).

### Example 2

### Characteristics of SIRCA Subjects

As described previously and in Example 1, the SIRCA sample was predominantly Caucasian (95%). Women were older than men as expected from enrollment criteria (see Tables 1A and 1B). Over 70% of these asymptomatic subjects had detectable CAC consistent with prevalent sub-clinical atherosclerosis and a recruitment strategy based on family history of premature heart disease (Tables 1A and 1B). Plasma resistin levels (median (IQR), ng/ml) were modestly but significantly higher in women than men (5.88 (4.42-7.84) versus 5.20 (3.87-6.90); p<0.001) (Tables 1A and 1B).

TABLE 1A
Characteristics of the Study Sample (preliminary)

| | | |
|---|---|---|
| | Men (n = 482) | Women (n = 414) |
| Characteristics | Median (IQR) | Median (IQR) |

| Age (years) | 46 | (41-52) | 50 | (44-57) |
|---|---|---|---|---|
| Total Cholesterol (mmol/L) | 5.16 | (4.51-5.80) | 5.47 | (4.74-6.09) |
| (mg/dL) | 199 | (174-224) | 211 | (183-235) |
| LDL Cholesterol (mmol/L) | 3.29 | (2.67-3.86) | 3.26 | (2.64-3.81) |
| (mg/dL) | 127 | (103-149) | 126 | (102-147) |
| HDL Cholesterol (mmol/L) | 1.09 | (0.93-1.27) | 1.48 | (1.19-1.76) |
| (mg/dL) | 42 | (36-49) | 57 | (46-68) |
| Triglycerides (mmol/L) | 1.45 | (1.03-2.06) | 1.28 | (0.90-1.68) |
| (mg/dL) | 128 | (91-182) | 113 | (80-149) |
| Glucose (mmol/L) | 5.28 | (4.89-5.78) | 5.11 | (4.78-5.5) |
| (mg/dL) | 95 | (88-104) | 92 | (86-99) |
| HOMA | 1.58 | (0.98-2.48) | 1.34 | (0.86-1.98) |
| Resistin (ng/ml) | 5.24 | (3.86-6.95) | 5.92 | (4.42-7.93) |
| Waist Circumference (cm) | 95.3 | (88.9-104.1) | 81.3 | (73.7-91.8) |
| Body Mass index (kg/m²) | 27.6 | (25.3-30.5) | 25.7 | (22.8-30.4) |
| Blood Pressure: systolic | 129 | (120-137) | 125 | (114-136) |
| Diastolic | 79 | (74-86) | 76 | (68-82) |
| Fasting glucose >126 mg/dl (%) | 8.1 | | 3.9 | |
| NCEP Metabolic Syndrome (%) | 31.5 | | 23.7 | |
| Smoking (%) | 12.7 | | 11.3 | |
| Medications: Statins (%) | 17.8 | | 10.4 | |
| Aspirin (%) | 18.5 | | 11.4 | |
| Hormone replacement therapy (%) | n/a | | 27.9 | |
| Coronary Artery Calcification | 136.0 ± 340.2 | | 44.0 ± 132.9 | |
| (CAC) Mean Score (±SD) | | | | |
| CAC Median (IQR) | 7 | (1-82) | 1 | (0-14) |

| | | |
|---|---|---|
| CAC > 70^{th} Percentile (%) | 40.9 | 39.1 |

TABLE 1B
Characteristics of the Study Sample (revised)

| | | |
|---|---|---|
| | Men (n = 471) | Women (n = 408) |
| Characteristics | Median (IQR) | Median (IQR) |

| Age (years) | 46 | (41-52) | 50 | (44-57) |
|---|---|---|---|---|
| Total Cholesterol (mmol/L) | 5.15 | (4.50-5.79) | 5.47 | (4.74-6.09) |
| LDL Cholesterol (mmol/L) | 3.28 | (2.66-3.85) | 3.26 | (2.64-3.80) |
| HDL Cholesterol (mmol/L) | 1.09 | (0.93-1.27) | 1.47 | (1.19-1.76) |
| Triglycerides (mmol/L) | 1.45 | (1.03-2.05) | 1.28 | (0.90-1.68) |
| Glucose (mmol/L) | 5.28 | (4.89-5.78) | 5.11 | (4.78-5.50) |
| Insulin | 44.7 | (29.6-67.0) | 37.9 | (26-1-57.1) |
| HOMA Index (units) | 1.57 | (0.98-2.43) | 1.32 | (0.85-1.96) |
| Resistin (ng/ml) | 5.24 | (3.86-6.95) | 5.92 | (4.42-7.93) |
| C Reactive Protein (mg/dl) | 1.1 | (0.5-2.1) | 1.5 | (0.6-3.7) |
| Soluble TNFα Receptor 2 (μg/ml) | 1.64 | (1.41-1.94) | 1.69 | (1.37-2.00) |
| Interleukin-6 (pg/ml) | 1.25 | (0.80-1.92) | 1.32 | (0.84-2.12) |
| Lp-PLA₂ (ng/ml) | 315 | (253-398) | 282 | (224-372) |
| Soluble ICAM-1 (ng/ml) | 295 | (260-332) | 286 | (258-323) |
| Waist Circumference (cm) | 95.3 | (88.9-104.1) | 81.3 | (73.7-91.8) |
| Body Mass index (kg/m²) | 27.6 | (25.3-30.5) | 25.7 | (22.8-30.4) |
| Blood Pressure: systolic | 129 | (120-137) | 125 | (114-136) |
| Diastolic | 79 | (74-86) | 76 | (68-82) |
| Fasting glucose >126 mg/dl (%) | 8.1 | | 3.9 | |
| Metabolic Syndrome (%)* | 30.4 | | 23.1 | |
| Smoking (%) | 12.7 | | 11.3 | |
| Medications: Statins (%) | 17.8 | | 10.4 | |
| Aspirin (%) | 18.5 | | 11.4 | |
| Hormone replacement therapy (%) | n/a | | 27.9 | |
| Coronary Artery Calcification | 130 ± 333 | | 42 ± 133 | |
| (CAC) Mean Score (±SD) | | | | |
| CAC Median (IQR) | 8 | (1-80) | 1 | (0-13) |

*Metabolic syndrome as defined by the National Cholesterol Education Program.
HOMA = homeostasis model assessment;
TNF = tumor necrosis factor;
Lp-PLA₂ = lipoprotein associated phospholipase A₂;
ICAM-1 = intercellular adhesion molecule-1.
To convert values for cholesterol to mg/dL, divide by 0.0259. To convert values for triglycerides to mg/dL, divide by 0.0113. To convert values for glucose to mg/dL, divide by 0.0555.

TABLE 2
Plasma Resistin Levels According to the Metabolic
Syndrome Features in SIRCA. Prevalence of Metabolic
Syndrome and Individual Components (preliminary)

| NCEP MetSyn | Plasma Resistin Levels Median (IQR) | | | | |
|---|---|---|---|---|---|
| Features | Absent | | Present | | P Value |
| Low HDL | 5.27 | (3.98-7.01) | 5.78 | (4.41-7.83) | <0.001 |
| Elevated | 5.59 | (4.22-7.59) | 5.32 | (3.92-7.07) | 0.14 |
| Triglycerides | | | | | |
| Elevated Fasting | 5.56 | (4.30-7.61) | 5.44 | (4.05-7.28) | 0.56 |
| Glucose | | | | | |
| High Blood Pressure | 5.47 | (4.14-7.23) | 5.64 | (3.92-7.88) | 0.50 |
| Central Obesity | 5.41 | (4.03-7.06) | 5.72 | (4.44-7.75) | 0.02 |
| NCEP MetSyn | 5.41 | (4.04-7.14) | 5.72 | (4.40-7.75) | 0.03 |
| Definition | | | | | |
| Upper quartile | 5.53 | (4.15-7.26) | 5.4 | (3.98-7.63) | 0.83 |
| HOMA | | | | | |
| WHO MetSyn | 5.48 | (4.15-7.23) | 5.41 | (3.93-7.70 | 0.95 |
| Definition | | | | | |

*Median plasma resistin levels were compared across metabolic syndrome features by Kruskal Wallis test.

### Example 3

### Association of Plasma Resistin with Inflammatory Factors in SIRCA

Plasma resistin levels were highly correlated with levels of diverse inflammatory markers, particularly sol TNF-R2, but also IL-6 and LpPLA_{2,} and to a lesser degree with sol ICAM-1 and CRP (see Tables 3A and 3B). Levels of sol TNF-R2 (p<0.001), LpPLA₂ (p=0.002) and IL-6 (p=0.04), but not CRP (p=0.2), remained positively associated with resistin in fully adjusted models: sol TNF-R2 levels were the strongest single predictor and accounted for 10% of variability in circulating resistin (Tables 4A and 4B). A scatterplot (data not shown) revealed that plasma resistin levels are correlated with log transformed plasma levels of soluble tumor necrosis factor (TNF) receptor 2 (Spearman R=0.31, p<0.001). The scatter plot showed an overlying linear regression line and 95% confidence interval (see Reilly et al, 2005, incorporated herein by reference). A second scatterplot (data not shown) indicated that plasma resistin levels are not correlated with the homeostasis model assessment index of insulin sensitivity (Spearman R=−0.003, p=0.93) (see Reilly et al, 2005, incorporated herein by reference).

Notably, resistin levels did not correlate with insulin resistance defined by the HOMA index (Tables 3A and 3B). In this regard, it is important to note that this study focuses on non-diabetic subjects of relatively normal weight (73% with BMI <30). However, consistent with previous reports (Yannakoulia et al, 2003; Azuma et al, 2003; Degawa-Yamauchi et al, 2003; Volarova de Courten et al, 2004, all cited above), SIRCA subjects with marked obesity (BMI >35; n=72) had a modest but significant increases in resistin levels compared to subjects with BMI <35[6.32 (4.38-8.76) versus 5.44 (4.12-7.23); p=0.04]. Similarly, SIRCA subjects with NCEP-defined metabolic syndrome (n=249) had slightly higher levels than subjects without the metabolic syndrome [5.72 (4.44-7.75) versus 5.41 (4.04-7.14); p=0.03]. Resistin levels also correlated inversely with HDL cholesterol in women (Tables 2A and 2B), but this was not significant in the adjusted analysis. Despite a trend towards gender differences in the strength of association with plasma resistin, there was no significant interaction of gender with inflammatory or metabolic factors in the relationship with resistin.

TABLE 3A
Correlation of Plasma Resistin Levels with Inflammatory,
Metabolic and Lipid Variables (preliminary)

| | Men | | Women | | All | |
|---|---|---|---|---|---|---|
| Variable | Rho | P | Rho | P | Rho | P |
| Sol-TNF-R2 | 0.26 | <0.001 | 0.36 | <0.001 | 0.31 | <0.001 |
| Interleukin-6 | 0.11 | 0.014 | 0.19 | <0.001 | 0.16 | <0.001 |
| LpPLA2-M | 0.19 | <0.001 | 0.12 | 0.02 | 0.13 | <0.001 |
| Hs-CRP | 0.04 | 0.42 | 0.11 | 0.03 | 0.10 | 0.003 |
| Sol-ICAM-1 | 0.09 | 0.09 | 0.11 | 0.03 | 0.09 | 0.01 |
| Plasma Insulin | −0.04 | 0.41 | 0.08 | 0.12 | 0.006 | 0.86 |
| HOMA | −0.05 | 0.31 | 0.07 | 0.13 | −0.003 | 0.93 |
| Plasma Glucose | −0.06 | 0.17 | 0.068 | 0.17 | −0.03 | 0.41 |
| Body Mass index | 0.036 | 0.43 | 0.09 | 0.09 | 0.034 | 0.30 |
| Waist circumference | 0.04 | 0.35 | 0.086 | 0.08 | 0.000 | 0.99 |
| HDL Cholesterol | −0.04 | 0.34 | −0.18 | 0.002 | −0.02 | 0.54 |
| Triglycerides | 0.002 | 0.97 | −0.015 | 0.77 | −0.01 | 0.68 |
| LDL Cholesterol | 0.01 | 0.85 | −0.07 | 0.16 | −0.035 | 0.31 |
| Systolic BP | 0.01 | 0.83 | −0.01 | 0.75 | −0.02 | 0.55 |

TABLE 3B
Correlation of Plasma Resistin Levels with Inflammatory,
Metabolic and Lipid Variables in SIRCA Subjects

| | Men (n = 471) | | Women (n = 408) | | All (n = 879) | |
|---|---|---|---|---|---|---|
| Variable | Rho | P | Rho | P | Rho | P |
| Waist circumference | 0.04 | 0.35 | 0.086 | 0.08 | 0.000 | 0.99 |
| Plasma Glucose | −0.06 | 0.17 | 0.068 | 0.17 | −0.03 | 0.41 |
| Plasma Insulin | −0.04 | 0.41 | 0.08 | 0.12 | 0.006 | 0.86 |
| HOMA Index | −0.05 | 0.31 | 0.07 | 0.13 | −0.003 | 0.93 |
| HDL Cholesterol | −0.04 | 0.34 | −0.18 | 0.002 | −0.02 | 0.54 |
| Triglycerides | 0.002 | 0.97 | −0.015 | 0.77 | −0.01 | 0.68 |
| LDL Cholesterol | 0.01 | 0.85 | −0.07 | 0.16 | −0.035 | 0.31 |
| CRP | 0.04 | 0.42 | 0.11 | 0.03 | 0.10 | 0.003 |
| Sol-TNF-R2 | 0.26 | <0.001 | 0.36 | <0.001 | 0.31 | <0.001 |
| Interleukin-6 | 0.11 | 0.014 | 0.19 | <0.001 | 0.16 | <0.001 |
| LpPLA2-M | 0.19 | <0.001 | 0.12 | 0.02 | 0.13 | <0.001 |
| Sol-ICAM-1 | 0.09 | 0.09 | 0.11 | 0.03 | 0.09 | 0.01 |

For Tables 3A, 3B: Spearman correlation coefficients are presented.
BP = blood pressure;
HOMA = homeostasis assessment;
CRP = high sensitivity C reactive protein;
Sol TNF-R2 = soluble tumor necrosis factor α receptor 2;
IL-6 = interleukin 6;
Lp-PLA2 = lipoprotein associated phospholipase A₂;
Sol ICAM-1 = soluble intercellular adhesion molecule-1.

TABLE 4A
Multivariable Analysis of Factors Associated with Plasma Resistin Levels (Preliminary)

| | Men | | Women | | All | |
|---|---|---|---|---|---|---|
| | Change in | | Change in | | Change in | |
| Factor | Resistin (CI) | P | Resistin (CI) | P | Resistin (CI) | P |
| Gender (M vs F) | — | — | — | — | −0.89 (0.83 to 0.96) | 0.002 |
| HDL Cholesterol | 1.02 (0.97 to 1.07) | 0.5 | 0.95 (0.93-0.98) | 0.003 | 0.98 (0.95 to 1.00) | 0.05 |
| (per 10 mg/dL) | | | | | | |
| Alcohol Intake | 0.90 (0.83 to 1.00) | 0.05 | 0.96 (0.88 to 1.05) | 0.38 | 0.93 (0.87 to 0.99) | 0.04 |
| (any vs none) | | | | | | |
| Ln-Sol TNF-αR2 | 1.57 (1.32 to 1.84) | <0.001 | 1.67 (1.43 to 1.95) | <0.001 | 1.62 (1.45 to 1.80) | <0.001 |
| (per log unit) | | | | | | |
| Ln-IL-6 | 1.07 (0.99 to 1.15) | 0.06 | 1.07 (1.01 to 1.24) | 0.73 | 1.06 (1.01 to 1.11−) | 0.013 |
| (per log unit) | | | | | | |
| Ln-LpPLA₂ | 1.21 (1.07 to 1.36) | 0.003 | 1.09 (0.96 to 1.24) | 0.2 | 1.14 (1.05 to 1.25) | 0.003 |
| (per log unit) | | | | | | |

Results of linear regression (natural log of resistin (In-resistin) as the dependent variable) are presented as the change In-resistin for a specific change in other variables.
*Nideks were adjusted for the following variables: age, systolic blood pressure, body mass index, HOMA, smoking (current vs. never and ex-smokers), exercise (none vs. any), HDL and LDL cholesterol, triglycerides, use of the following medications (statins, aspirin, and hormone replacement therapy (HRT) in women) and plasma hs-CRP levels.

TABLE 4B
Multivariable Analysis of Factors Associated with Plasma Resistin Levels in SIRCA

| | Men | | Women | | All | |
|---|---|---|---|---|---|---|
| | Change in | | Change in | | Change in | |
| Factor | Resistin (CI) | P | Resistin (CI) | P | Resistin (CI) | P |
| Gender (M vs F) | — | — | — | — | −0.59 (−1.01 to −0.16) | <0.001 |
| Ln-Sol TNF-R2 | 3.05 (2.30 to 3.81) | <0.001 | 3.16 (2.11 to 4.21) | <0.001 | 3.05 (2.30 to 3.80) | <0.001 |
| (per log unit) | | | | | | |
| Ln-IL-6 | 0.33 (−0.10 to 0.762) | 0.30 | 0.54 (0.11 to 1.44) | 0.001 | 0.33 (0.03 to 0.63) | 0.04 |
| (per log unit) | | | | | | |
| Ln-LpPLA₂ | 0.83 (0.04 to 1.63) | 0.002 | 0.40 (−0.05 to 1.30) | 0.17 | 0.64 (0.04 to 1.23) | 0.002 |
| (per log unit) | | | | | | |

Results are presented as the change in plasma resistin level (ng/ml) for a specific change in other factors. Because plasma resistin levels were not normally distributed, the linear regression model used natural log of resistin as the dependent variable.
Models were adjusted for the following variables; age, systolic blood pressure, body mass index, HOMA, smoking, exercise, HDL and LDL cholesterol, triglycerides, CRP levels, and use of the following medications (statins, aspirin, and hormone replacement therapy (in women)).
There was no significant interaction, by the likelihood ratio tests, of gender with any metabolic or inflammatory factor (all p >0.1) in the association with plasma resistin levels.
Sol TNF-R2 = soluble tumor necrosis factor receptor 2;
IL-6 = interleukin 6;
LpPLA₂ = lipoprotein associated phospholipase A₂;
Ln = natural log transformation/
CI = 95% confidence interval.

### Example 4

### Plasma Resistin Levels in Type 2 Diabetics and Young Healthy Subjects

In the type 2 diabetic sample, resistin levels [median (IQR), ng/ml] tended to be higher in women [5.98 (3.42-7.89)] than men 5.76 (4.29-7.95) in men] and tended to be higher than in our SIRCA sample. Remarkably, as in SIRCA, resistin levels were strongly associated with plasma sol TNF-R2 (p<0.001) but were not significantly correlated to measures of adiposity and insulin resistance (Table 5). In fact, in multivariable analysis, only plasma levels of sol TNF-R2 (p<0.001) and the white cell count (p=0.013) were independent predictors of log-transformed plasma resistin levels.

In young healthy subjects, plasma resistin levels, e.g., at 6 am, 3.73 (2.50 to 4.58); at 12 noon, 3.65 (2.10 to 3.94); at 6 pm, 3.22 (2.27 to 4.24); and at 6 am next morning, 3.15 (2.27 to 3.59), tended to be lower than in SIRCA and were remarkably stable over a 24 hour period (repeated measures ANOVA F statistic for time=1.15, p=0.36).

TABLE 5
Correlation of Plasma Resistin Levels with Inflammatory,
Metabolic and Lipid Variables in Type 2 Diabetic Subjects

| | Men (n = 167) | | Women (n = 48) | | All (n = 215) | |
|---|---|---|---|---|---|---|
| Variable | Rho | P | Rho | P | Rho | P |
| Waist circumference | 0.00 | 0.99 | 0.18 | 0.2 | 0.05 | 0.43 |
| Plasma Glucose | −0.12 | 0.12 | 0.25 | 0.09 | 0.04 | 0.50 |
| Plasma Insulin | −0.10 | 0.22 | 0.12 | 0.41 | −0.03 | 0.63 |
| Plasma Leptin | 0.05 | 0.47 | 0.15 | 0.29 | 0.06 | 0.39 |
| HDL Cholesterol | 0.00 | 0.99 | −0.11 | 0.50 | −0.04 | 0.59 |
| Triglycerides | 0.09 | 0.27 | 0.28 | 0.05 | 0.12 | 0.09 |
| LDL Cholesterol | −0.04 | 0.60 | 0.30 | 0.04 | 0.04 | 0.53 |
| Sol-TNF-R2 | 0.37 | <0.001 | 0.42 | 0.003 | 0.38 | <0.001 |
| CRP | 0.10 | 0.18 | 0.12 | 0.41 | 0.11 | 0.11 |
| White Cell Count | 0.17 | 0.02 | −0.08 | 0.6 | 0.12 | 0.09 |

Spearman correlation coefficients are presented.
CRP = high sensitivity C reactive protein;
Sol TNF-R2 = soluble tumor necrosis factor α receptor 2.

### Example 5

### Association of Plasma Resistin Levels with CAC in SIRCA

Risk factors that are associated with CAC in the SIRCA sample include age, gender, adiposity, LDL cholesterol, HDL cholesterol, smoking, systolic blood pressure, plasma glucose and use of statins. The metabolic syndrome, but not CRP levels, is strongly associated with CAC in this sample. See, Reilly et al, 2004b, 2004a and 2003a, all cited above.

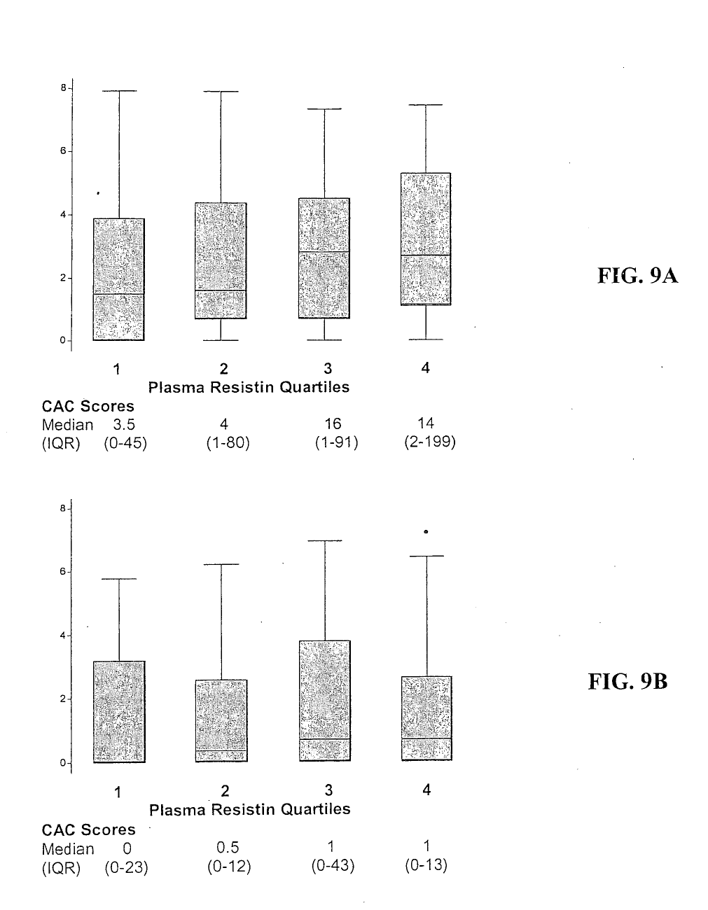

Median (IQR) CAC scores increased across plasma resistin quartiles in men (p=0.01) and women (p=0.05) (FIGS. 9A and 9B). There was no significant interaction (LR test p=0.8) between gender and plasma resistin levels in the association with CAC. Therefore, results of multivariable analyses are presented for men and women combined. Resistin levels were associated with CAC after controlling for age, gender, and established risk factors and even with further adjustment for the metabolic syndrome and CRP levels (Tables 6A and 6B). Adding plasma resistin levels to a fully adjusted multivariable model containing plasma CRP levels (LR test p=0.04) strengthened the association with CAC scores whereas CRP did not add significantly to a model that already contained plasma resistin levels (LR test p=0.2). There was no statistically significant interaction between gender and plasma resistin levels in the association with CAC. These Tables demonstrate that serum resistin level is a significant risk factor for coronary artery calcification (CAC) in humans. The two entries including Met Syn show that elevated resistin imparts a 25% increased risk for CAC, which is an accepted surrogate for atherosclerosis, even in patients with metabolic syndrome and even after accounting for CRP levels.

TABLE 6A
Association of Plasma Resistin Levels with Coronary
Artery Calcification (CAC) in Multivariable Ordinal
Logistic Regression (Preliminary)

| | Odds Ratio(CI) | | |
|---|---|---|---|
| Adjusted For | in Men | | P (Men) |
| Age, gender | 1.23 | (1.03 to 1.47) | 0.02 |
| Age, Gender, RF* | 1.23 | (1.02 to 1.47) | 0.03 |
| Age, Gender, RF*, CRP | 1.23 | (1.03 to 1.52) | 0.02 |
| Age, Gender, RF*, CRP, MetSyn | 1.25 | (1.04 to 1.50) | 0.01 |
| Age, Gender, RF*, CRP, MetSyn, | 1.23 | (1.02 to 1.47) | 0.03 |
| and In-HOMA | | | |

Odds ratio and 95% confidence interval (CI) for increase in CAC category for a 5 ng/ml increase in plasma resistin levels. CAC categories used in ordinal regression were CAC 0, CAC 1-10, CAC 11-100, CAC >100.
*Risk factors (RF*) included total cholesterol, HDL cholesterol, systolic blood pressure, cigarette smoking, exercise, alcohol, race, family history of premature CAD, and medication use (aspirin, statin, beta blocker, and hormone replacement therapy in women).
Ln-HOMA is the natural log transformation of HOMA values.

TABLE 6B
Association of Plasma Resistin Levels with
Coronary Artery Calcification (CAC) in SIRCA

| | | |
|---|---|---|
| Adjusted For | Odds Ratio (CI) | P |

| | | | |
|---|---|---|---|
| Age, gender | 1.23 | (1.03 to 1.47) | 0.02 |
| Age, Gender, RF* | 1.23 | (1.02 to 1.47) | 0.03 |
| Age, Gender, RF†, MetSyn | 1.25 | (1.04 to 1.50) | 0.01 |
| Age, Gender, RF†, MetSyn, | 1.23 | (1.02 to 1.47) | 0.03 |
| and CRP | | | |

Odds ratio and 95% confidence interval (CI) for increase in CAC category for a 5 ng/ml increase in plasma resistin levels. CAC categories used in ordinal regression were CAC 0, CAC 1-10, CAC 11-100, CAC >100.
*Risk factors (RF*) included total cholesterol, HDL cholesterol, systolic blood pressure, cigarette smoking, exercise, alcohol, race, family history of premature CAD, and medication use (aspirin, statin, beta blocker, and hormone replacement therapy in women).
In models that contained metabolic syndrome, non metabolic syndrome risk factors (RF†) were smoking, exercise, alcohol intake, race, LDL cholesterol, and medication use.
CRP = C reactive protein.
There was no statistically significant interaction between gender and plasma resistin levels in the association with CAC.

In multivariable models adjusted for age, gender and non metabolic syndrome risk factors, plasma levels of resistin were significantly associated with CAC in subjects with the metabolic syndrome (p=0.003) (see Tables 7A and 7B). By contrast, in this sample CRP levels were not predictive of CAC independent of metabolic syndrome (p=0.65).

TABLE 7A
Association of Plasma Resistin Levels with Coronary Artery
Calcification (CAC) by Risk Factor Categories (Preliminary)

| Category | Odds Ratio (CI) | | Interation P |
|---|---|---|---|
| LDL Cholesterol ≦130 mg/dL | 1.20 | (0.94 to 1.54) | 0.4 |
| LDL Cholesterol >130 mg/dL | 1.36 | (1.03 to 1.80) | |
| Framingham Risk Score | 1.23 | (1.02 to 1.47) | 0.03 |
| Hs-CRP <1.0 mg/dL | 1.34 | (1.02 to 1.88) | 0.05 |
| Hs-CRP 1-3 mg/dL | 1.21 | (0.88 to 1.64) | |
| Hs-CRP >3 mg/dL | 1.17 | (0.83 to 1.63) | |
| NCEP Metabolic Syndrome | 1.11 | (0.90 to 1.37) | 0.04 |
| absent | | | |
| NCEP Metabolic Syndrome | 1.81 | (1.24 to 2.66) | |
| present | | | |
| LpPLA2 <75^{th} percentile | 1.16 | (0.92 to 1.45) | 0.02 |
| LpPLA2 >75^{th} percentile | 2.01 | (1.36 to 3.00) | |

Odds ratio and 95% confidence interval (CI) for increase in CAC category for a 5 ng/ml increase in plasma resistin levels. CAC categories used in ordinal regression were CAC 0, CAC 1-10, CAC 11-100, CAC >100.
*Risk factors (RF*) included in the models include total cholesterol, HDL cholesterol, systolic blood pressure, cigarette smoking, exercise, alcohol, race, family history of premature CAD, and medication use (aspirin, statin, beta blocker, and hormone replacement therapy in women).
The NCEP Met Syn entries demonstrate that increased serum resistin level is not much of a risk factor in patients without the metabolic syndrome (obesity, insulin resistance, but is a large risk factor (81% increase) in patients with metabolic syndrome.

TABLE 7B
Association of Plasma Resistin Levels and C Reactive
Protein Levels with Coronary Artery Calcification
(CAC) in SIRCA Metabolic Syndrome Subgroups

| Metabolic | Resistin | | | C Reactive Protein | | |
|---|---|---|---|---|---|---|
| Syndrome | Odds Ratio (CI) | | P Value | Odds Ratio (CI) | | P value |
| Absent | 1.11 | (0.90 to 1.37) | 0.44 | 1.04 | (0.97 to 1.12) | 0.14 |
| Present | 1.81 | (1.24 to 2.66) | 0.003 | 1.01 | (0.92 to 1.11) | 0.65 |

| | | |
|---|---|---|
| Interaction P | 0.03 | 0.70 |

Odds ratio and 95% confidence interval (CI) for increase in CAC category for a 5 ng/ml increase in plasma resistin levels and a 1 mg/dl increase in C reactive protein (CRP) level in ordinal regression models adjusted for age, gender, smoking, exercise, alcohol intake, race, LDL cholesterol, and medication use (aspirin, statin, beta blocker, and hormone replacement therapy in women).
*The likelihood ratio test was used to test for interaction of resistin with metabolic syndrome subgroups in the association with CAC.

### Example 6

### Protocols for Identifying the Inflammatory Cascade Leading to Hyperresistinemia in Humans

A. Differentiation of Primary Human Macrophages.

Peripheral blood mononuclear cells were isolated from whole blood of healthy donors following apheresis and elutriation. Greater than 90% of these monocytes expressed CD14 and HLA-DR. Cells were plated in 24 well plates at a density of 10⁶ cells per well, allowed to adhere for 4 hours, then washed with Dulbecco's Modified Eagles Medium (DMEM) and further cultured in 10% FBS in DMEM supplemented with 5 ng/ml GM-CSF (Sigma) to promote macrophage differentiation. All experiments were performed after overnight equilibration with macrophage serum free medium (Gibco/Invitrogen) supplemented with 5 ng/ml GM-CSF. Cells were treated as indicated with LPS (Sigma), aspirin (Sigma), SN50 and control peptide (Biomol), MG132, PD98059, SB20358 (Calbiochem), and TNFα (R&D Systems). Neutralizing antibodies to TNFα, IL-6, and anti-IL-6α, as well as control IgG were obtained from R&D Systems. Adenovirus expressing activated IKK in pAD-easy™ vector with GFP and control vector were a generous gift from Steven Shoelson.

B. RNA Isolation and Quantification.

RNA was isolated using RNeasy® Mini Kit (Qiagen), then subjected to DNAse digestion followed by reverse transcription (Invitrogen). mRNA transcripts were quantified by the dual-labeled fluorogenic probe method for real time PCR, using a Prism® 7900 thermal cycler and sequence detector (Perkin Elmer/ABI). Real time PCR was performed by using Taqmang Universal Polymerase Master Mix (Applied Biosystems). The primers and probes used in the real-time PCR were the following:

| | |
|---|---|
| Sense-Resistin: | |
| 5-AGCCATCAATGATAGGATCCA-3; | SEQ ID NO: 1 |
| Antisense-Resistin: | |
| 5-TCCAGGCCAATGCTGCTTAT-3;, | SEQ ID NO: 2 |
| Resistin Probe: | |
| 5-Fam-AGGTCGCCGGCTCCCTAATATTTAGGGTAMR | SEQ ID NO: 3 |
| A-3, | |
| Sense human 36B4 sense, | |
| 5′-TCGTGGAAGTGACATCGTCTTT-3′; | SEQ ID NO: 4 |
| Antisense 36B4, | |
| 5′-CTGTCTTCCCTGGGCATCA-3′; | SEQ ID NO: 5 |
| and | |
| 36B4 Probe | |
| 5′-FAM-TGGCAATCCCTGACGCACCG-TAMRA-3′. | SEQ ID NO: 6 |

Primer and probe for TNFα was obtained from ABI. The cycle number at which the transcripts of the gene of interest was detectable (CT) was normalized to the cycle number of 36B4 detection, referred to as δ CT. The fold change of the gene of interest expression relative to the vehicle treated group was expressed as 2⁻ δδ^{CT}, in which δδ CT equals the δCT of the compound treated group minus δCT of the chosen control group, which was normalized to 1.

C. ELISA

Resistin concentrations, in cell media and human plasma, were assessed with a commercially available ELISA (Linco) and normalized to cell protein. Average correlation coefficient for standards using 4 parameter fit was 0.99. Intra-assay and inter-assay coefficients of variance were 4.7% and 9.1%, respectively. Direct comparison of standard curves generated by the Linco kit with another commercially available resistin ELISA (Biovendor) yielded high correlation (rho=0.99, p<0.001), except that the Biovendor results were approximately 30% lower than those determined with the Linco assay. This appeared to be related to the standards used for calibration. Discrepant absolute values among different assays, including the Biovendor assay, were recently described (Pfutzner 2003 cited above). Resistin levels in 40 plasma samples were measured using both Linco and Biovendor ELISA kits, with moderate correlation (rho=0.66). Levels of soluble TNFα Receptor-2 were measured using a commercially available immunoassay (R&D Systems). Intra-assay and inter assay coefficients of variance were 5.1% and 9.8%, respectively.

D. Human Endotoxemia Study.

Healthy volunteers (n=6, 3 male, 3 female) aged 18-45 with BMI between 20 and 30 and on no medications were studied. The University of Pennsylvania Institutional Review Board approved the study protocol and all subjects gave written informed consent. Following screening and exclusion of subjects with any clinical or laboratory abnormalities, subjects were admitted to the General Clinical Research Center at the University of Pennsylvania for a 60 hour stay. Serial blood samples were collected for 24 hours prior to and 24 hours following the intravenous administration of human research grade endotoxin [obtained from NIH Clinical Center, reference endotoxin (CCRE) (lots 1 and 2; NIHCC PDS #67801)] at a dose of 3 ng/kg given at 6 AM. Plasma and whole blood RNA (PAX tube isolators, Qiagen) samples were isolated from blood, and stored under appropriate conditions for subsequent assays.

E. Type 2 Diabetes Study.

Subjects with type 2 diabetes (n=215, men=167, women=48), aged 35-75 and free from clinical cardiovascular diseases (CVD), were recruited through the diabetes clinics at the University of Pennsylvania Medical Center and the Veterans Affairs Medical Center, Philadelphia, to an ongoing study of cardiovascular risk factors in type 2 diabetes. The sample was composed of 59% Caucasians and 35% African-Americans. All subjects were evaluated at the University of Pennsylvania General Clinical Research Center (GCRC) in a fasting state at 8 AM. The University of Pennsylvania Institutional Review Board approved the study protocol and all subjects gave written informed consent. The patient population is described in Reilly et al, 2004c, cited above.

F. Statistical Methods.

Data were reported as mean and standard error (SE) for continuous variables. Because of baseline variation in cell populations between batches of primary human monocytes isolated from multiple different donors, cell culture experiments were performed in triplicate and data from representative experiments are presented. For cell culture experiments with multiple treatments, analysis of variance (ANOVA) was used to test for differences in means across treatment groups. When significant global differences were found, post-hoc t-tests were applied to compare specific treatment groups to the control. Data from the human endotoxemia experiment were analyzed by repeated measures ANOVA. In the type 2 DM human study, Spearman correlations of plasma levels of resistin with plasma sTNF-R2 levels are presented.

### Example 7

### Induction of Resistin Gene and Protein Expression by Endotoxin Treatment of Human Macrophages

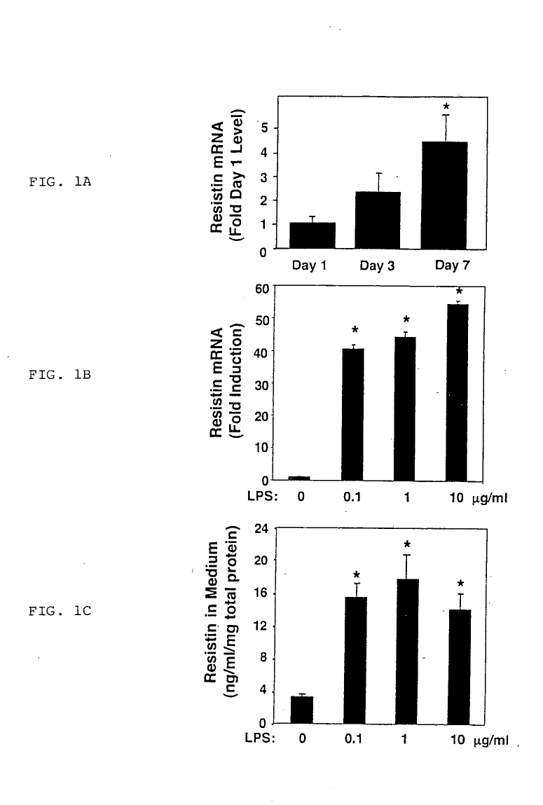

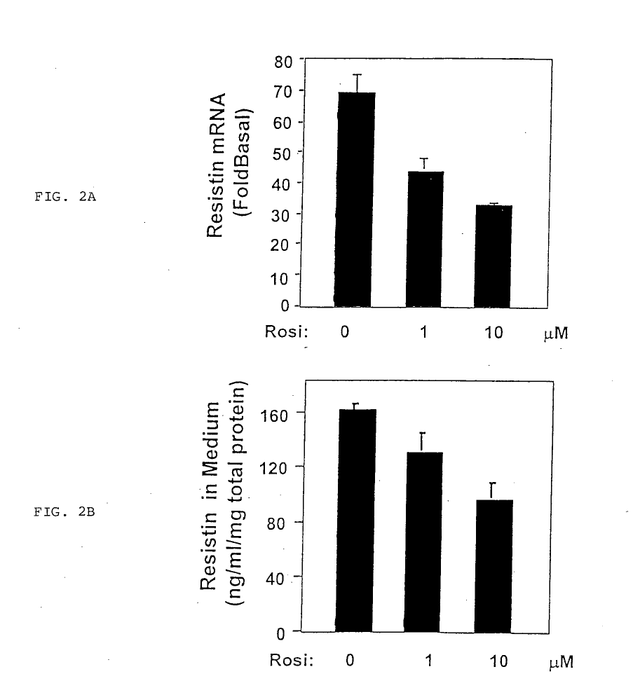

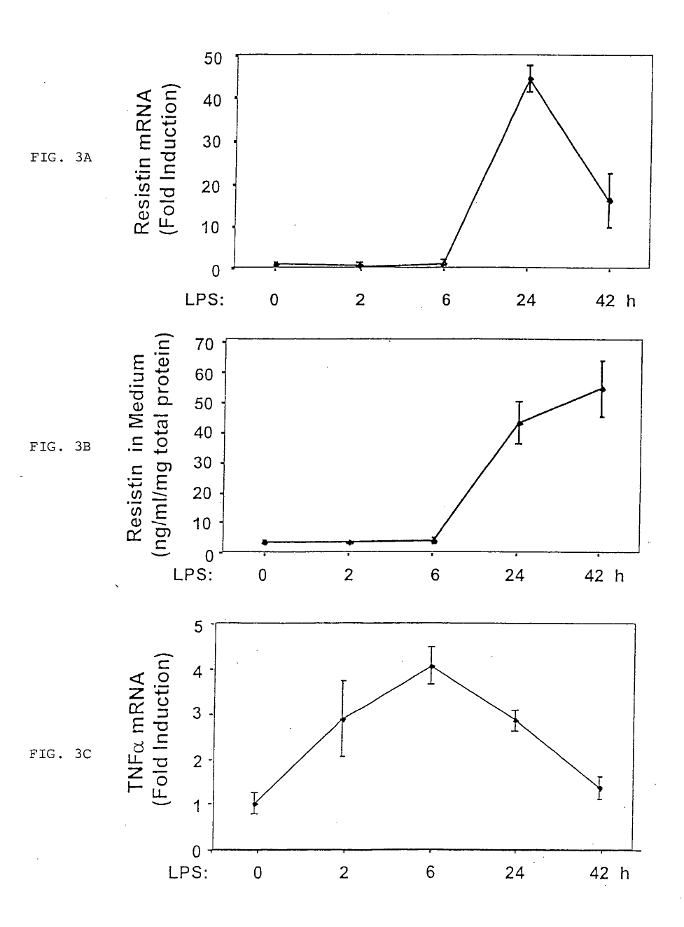

The regulation of resistin expression was studied in primary cultures of human monocytic cells. Immediately upon plating of elutriated primary human monocytes, resistin gene expression was detectable but highly variable from experiment to experiment (data not shown). One day after plating, resistin gene expression remained detectable at low levels (FIG. 1A). Subjection of the cells to a protocol leading to differentiation along the macrophage lineage led to a modest, time-dependent enhancement of resistin gene-expression (FIG. 1A). In agreement with a previous report of Kaser et al, 2003, cited above, treatment of primary macrophages with the endotoxin LPS led to a dramatic, dose-responsive increase in resistin gene expression (FIG. 1B). This effect of LPS was paralleled by an increase in resistin protein secretion into the medium (FIG. 1C). Of note, activated mouse peritoneal macrophages harvested after thioglycolate treatment did not express detectable levels of mouse resistin, even after treatment with LPS (data not shown).

### Example 8

### Endotoxin Induction of Resistin is Delayed with Respect to TNFα

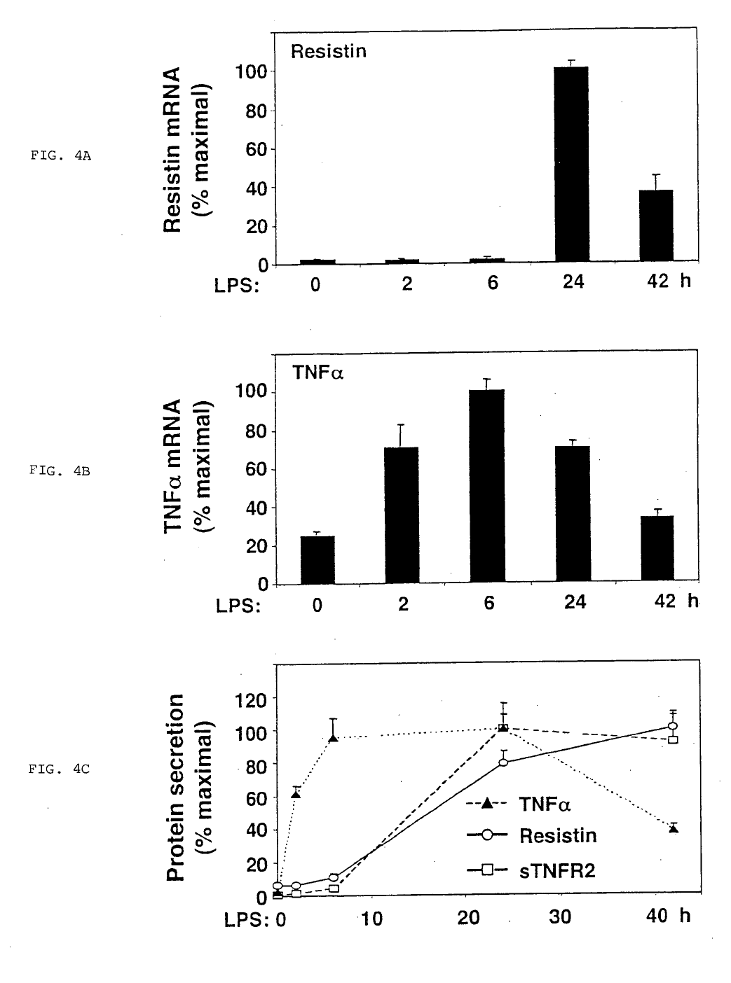

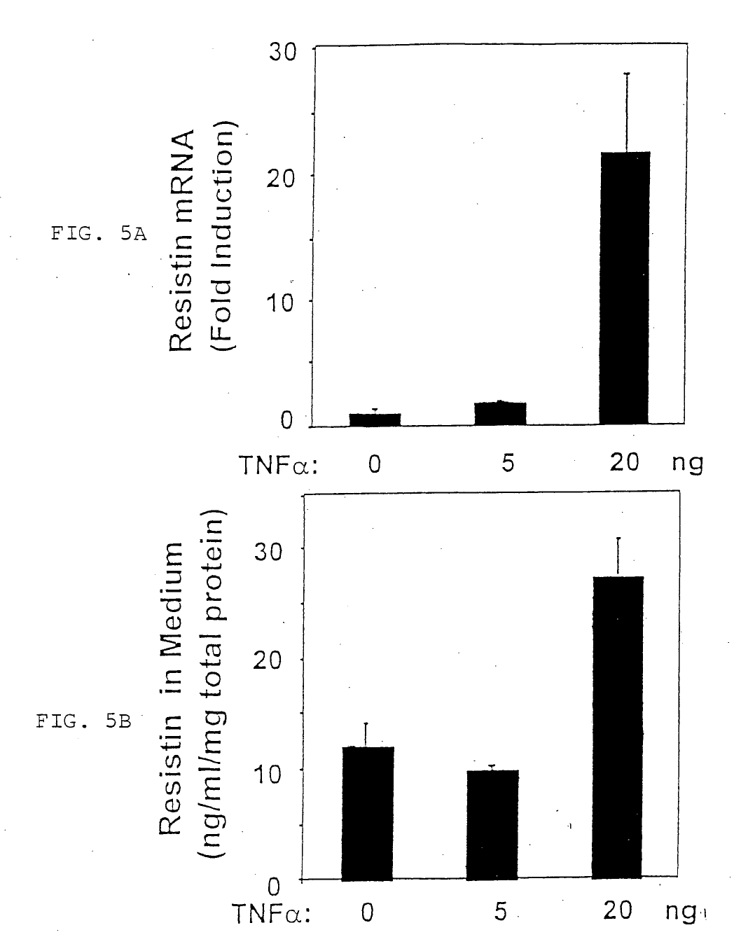

Induction of resistin gene expression by LPS exposure of human macrophages began between 6 and 24 hours after treatment, with peak expression at 24 hours (FIG. 4A). This time-course of resistin induction was delayed relative to induction of TNFα gene expression, which was detectable at 2 hours and peaked 6 hours after LPS exposure (FIG. 4B). The secretion of TNFα followed a similar time course (FIG. 4C). By contrast, secretion of resistin did not increase until much later, more closely following that of the appearance of soluble TNF receptor 2 (sTNFR2), a marker of TNFα action (FIG. 4C) (Idress et al, 2000 Microsc. Res. Tech., 50:184-195).

### Example 9

### Endotoxin Induction of Resistin is Blocked by Immunoneutralization of Multiple Cytokines

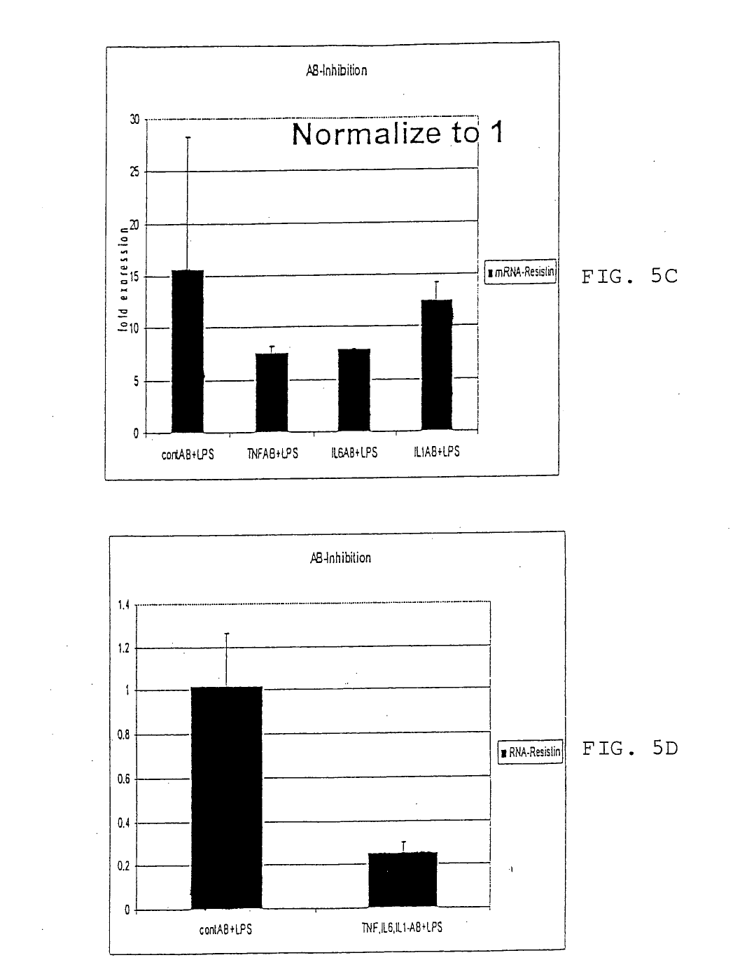

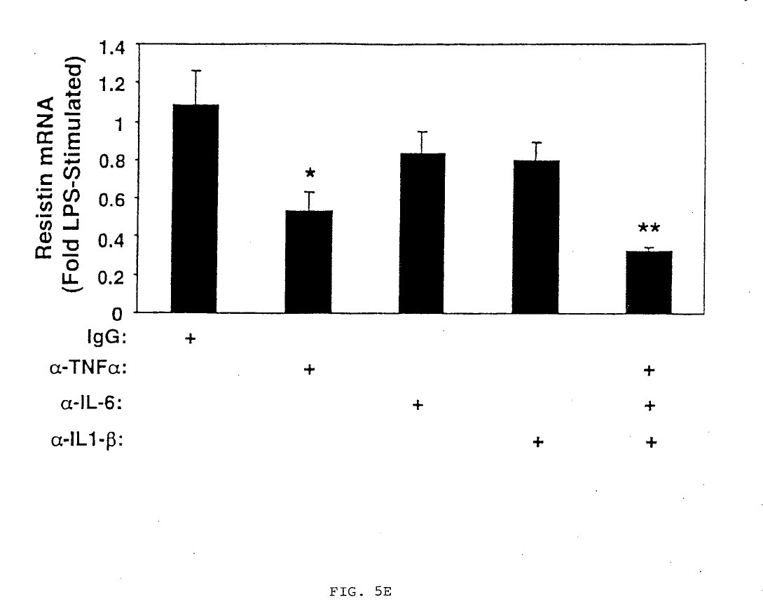

Resistin gene expression was also induced by TNFα treatment of primary human macrophages (FIG. 5A) (Kaser et al, 2003, cited above) and resistin secretion increased in parallel (FIG. 5B). Since LPS induction of TNFα preceded the increase in resistin (FIG. 4C), TNFα, or a similar cytokine produced early after LPS exposure was theorized to be responsible for the later induction of resistin. Indeed, neutralizing antibodies to TNFα markedly attenuated the increase in resistin gene expression (FIG. 5E). LPS treatment also induces other cytokines, including interleukin-6 (IL-6) and interleukin-la (IL-6α) (Van Amersfoort et al, 2003 Clin. Microbiol. Rev. 16:379-414), and IL-6 induces resistin modestly (data not shown and Kaser et al, 2003 cited above). Antibodies to IL-6 and IL1—individually had minor effects on LPS-stimulation of resistin (FIG. 5E). However, the combination of antibodies to TNFα, IL-6, and IL-6α markedly attenuated LPS induction of resistin (FIG. 5E). These data clearly show that resistin induction by endotoxin is mediated by a cascade in which the primary event is secretion of inflammatory cytokines that, in turn, induce resistin.

### Example 10

### Induction of Resistin is Blocked by Anti-Inflammatory Insulin Sensitizing Drugs that Target NF-κB

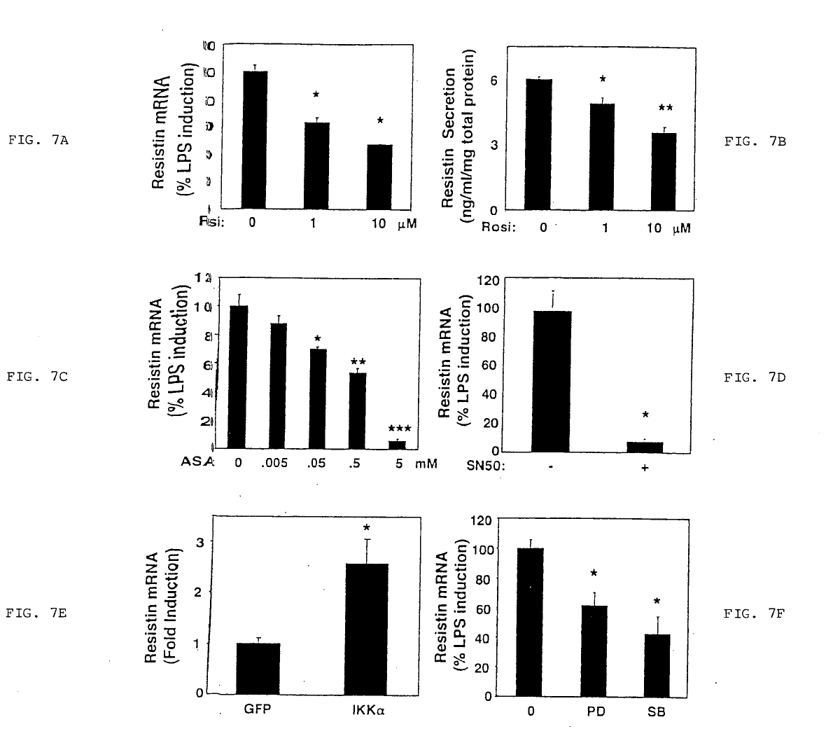

Mouse resistin, produced exclusively by adipocytes, is down-regulated by antidiabetic thiazolidinediones (TZD) including rosiglitazone. Consistent with an earlier report (Patel et al, 2003 cited above) rosiglitazone down-regulated resistin gene expression (FIG. 7A) in LPS-stimulated human macrophages. Resistin protein secretion was also significantly reduced by rosiglitazone (FIG. 7B). Hence, macrophage expression of resistin and its induction by LPS is species-specific, but down-regulation of resistin by TZD occurs both in rodents and humans. Rosiglitazone has marked anti-inflammatory effects on macrophages. This led to the examination of the effect of aspirin, an anti-inflammatory compound that targets IκB kinase and has insulin sensitizing effects (Yuan et al, 2001 Sci. 293:1673-77). Remarkably, aspirin dramatically decreased endotoxin-induced resistin expression in a dose-dependent manner (FIG. 7C). Both aspirin (via IκB kinase) and rosiglitazone (via PPARγ) inhibit NF-κB (Ricote et al 1998 Nature, 391:79-82; Yuan, cited above) which is activated by LPS. Indeed, treatment of the macrophages with the proteasome inhibitor MG132, which prevents NF-κB activation, abrogated endotoxin-induced activation of resistin expression (data not shown). Moreover, treatment of the macrophages with SN50, a cell-permeable peptide that specifically prevents activation of NF-κB by inhibiting its nuclear translocation (Lin et al, 1995 J. Biol. Chem., 270:14255-58) nearly abolished endotoxin-induced activation of resistin expression (FIG. 7D).

Thus, activation of NF-κB is required for LPS induction of resistin in human macrophages. Furthermore, constitutive activation of NF-κB by adenoviral expression of activated IκB kinase was sufficient to induce resistin in primary human macrophages (FIG. 7E). The magnitude of this activation was less than that caused by LPS, which is known to also activate MAP-kinase. Indeed, inhibition of either p42 MAPK by PD98059, or p38 MAPK (using SB20358) partially blocked the induction of resistin by LPS (FIG. 7F). Together these results show that NF-κB activation is necessary and sufficient for resistin induction by LPS, with MAP-kinase activation increasing the magnitude of the response.

### Example 11

### LPS Robustly Increases Circulating Resistin Levels in Normal Humans

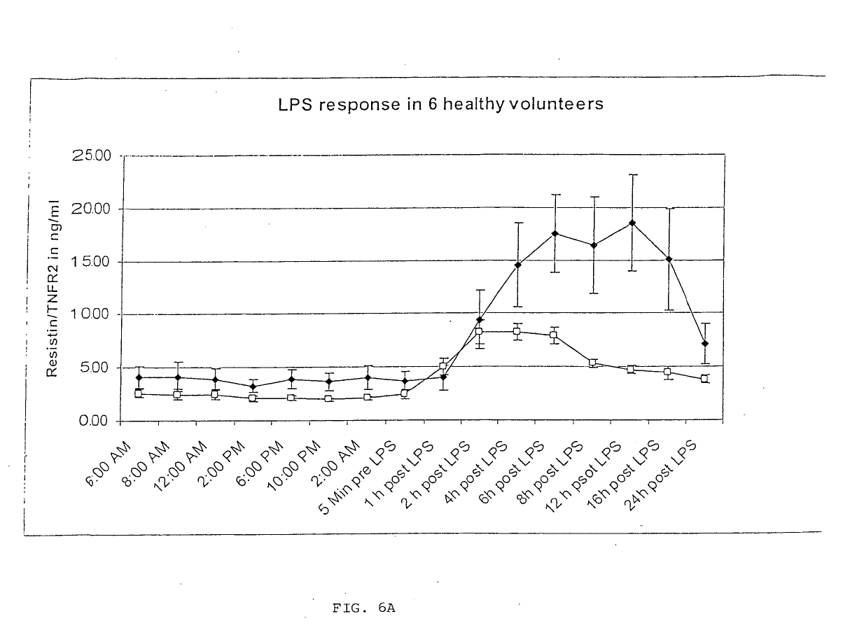

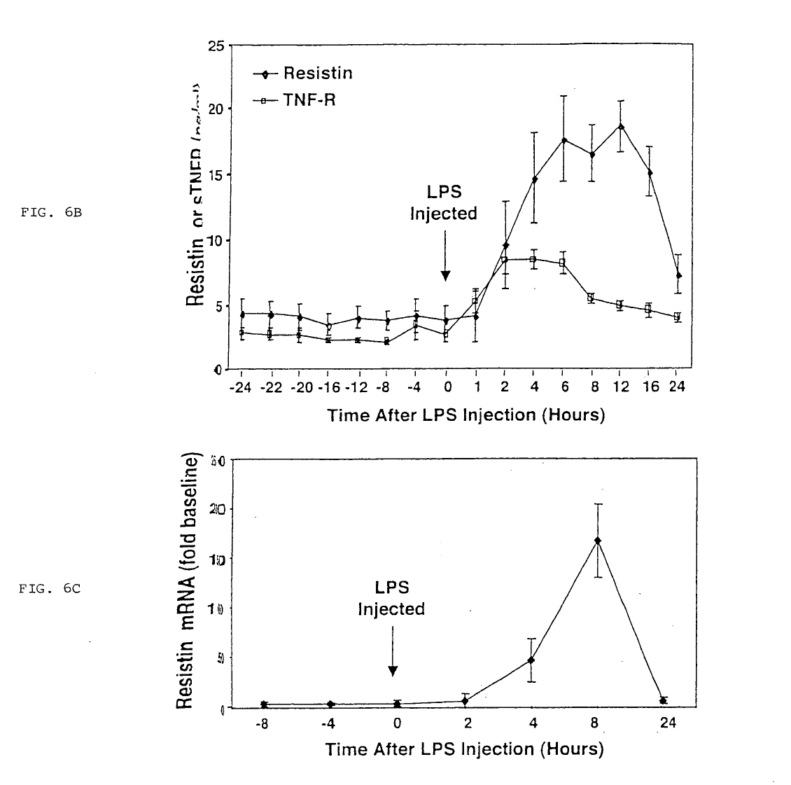

To determine if these findings from ex vivo studies of human macrophages in the preceding examples translated into in vivo observations in humans, six normal volunteers were injected with LPS, using a protocol similar to that shown to produce insulin resistance (Scoop et al 2002 Am. J. Physiol. Endocrinol. Metab., 282:E1276-85). At baseline, circulating resistin levels were ˜4 ng/ml, and remained relatively constant for several hours prior to LPS infusion (FIG. 6B). Remarkably, resistin levels rose dramatically due to endotoxemia, peaking 8-16 hours after LPS administration (FIG. 6B). The time course of hyperresistinemia paralleled the increase in circulating levels of sTNFR2, although the increase in resistin levels was more marked and sustained (FIG. 6B). The increase in resistin protein levels correlated with increased resistin gene expression in peripheral blood mononuclear cells following systemic endotoxemia (FIG. 6C).

### Example 12

### Circulating Resistin Levels Correlate with the Inflammatory Marker STNFR2 in Patients with Type 2 Diabetes

Patients with Type 2 diabetes and insulin resistance, many of whom are obese, have elevated levels of several inflammatory markers including IL-6 and TNFα, and sTNFR. LPS, administration has been shown to induce acute insulin resistance in humans. Given that LPS infusion increased resistin levels, resistin was measured in a cohort of 215 patients with type 2 diabetes. Circulating serum and plasma resistin levels were significantly correlated with levels of soluble TNF receptor 2 in human patients with type 2 diabetes. Scatterplots (data not shown) showed the correlation (Spearman coefficient rho=0.38 (p<0.001) of plasma resistin and soluble TNF receptor 2 levels in 215 humans with type 2 diabetes. (see Reilly et al, 2005, cited above, incorporated by reference). Thus, there is an association between resistin levels and systemic inflammation in patients with type 2 diabetes.

The line represents the linear regression fit between log transformed plasma levels of resistin and sTNFR2.

All documents, including the priority document, cited within this specification are incorporated herein by reference.

1. A method of determining the risk or progression of a cardiovascular disease comprising
measuring the level of resistin protein in a biological fluid of a mammalian subject;
comparing the subject's level to a standard of resistin levels in a population wherein an elevated resistin level compared to said standard is predictive of increased risk of disease.

2. The method according to claim 1, wherein said population is comprised of healthy subjects and subjects with cardiovascular disease.

3. The method according to claim 1, wherein a resistin level greater than that of the lowest 25% of the resistin levels forming the population is indicative of risk of cardiovascular disease.

4. The method according to claim 1, wherein a resistin level greater than that of 50% of the resistin levels forming the population is indicative of an intermediate risk of cardiovascular disease.

5. The method according to claim 1, wherein a resistin level greater than that of 75% of the resistin levels forming the population is indicative of high risk of cardiovascular disease or progression of existing cardiovascular disease.

6. The method according to claim 1, wherein the resistin level is greater than that of 80% over the normal range and is indicative of highest risk of disease.

7. The method according to claim 1, wherein the measuring comprises contacting a sample of the subjects' serum with an anti-resistin antibody and detecting the concentration of serum resistin-antibody complex in the sample.

8. The method according to claim 1, further comprising further measuring the concentration of a second biomarker of cardiovascular disease or a second inflammatory biomarker in the sample and correlating the resistin level with the level of the second biomarker, wherein the combination of resistin concentration and second biomarker concentration is predictive of cardiovascular risk.

9. The method according to claim 1, wherein the measurement is taken repeatedly over time to monitor the progression of cardiovascular disease risk over time.

10. The method according to claim 1, wherein the subject is selected from the group consisting of a subject asymptomatic for cardiovascular disease and not diabetic, a subject symptomatic for cardiovascular disease, a subject symptomatic for metabolic syndrome, a subject who is a diabetic, and a subject having type 2 diabetes.

11. The method according to claim 1, wherein said disease is atherosclerosis.

12. A method of treating or retarding the progress of an inflammatory disorder or a cardiovascular disorder in a mammalian subject comprising reducing the level or effect of the subject's circulating resistin.

13. The method according to claim 12, comprising reducing the levels by at least 10-20% of presenting levels.

14. The method according to claim 12, further comprising measuring said subject's resistin levels by comparing the subject's level to a standard of resistin levels in a population, and reducing said subject's level to a level less than that of the top 20% of said standard.

15. The method according to claim 13, further comprising reducing said subject's level to a level less than that of the top 25% of said standard.

16. The method according to claim 13, further comprising reducing said subject's level to a level less than that of the top 50% of said standard.

17. The method according to claim 13 comprising said subject's level to a level less than that of the lowest 25% of said standard.

18. The method according to claim 12, wherein the reducing comprises administering an amount of an antagonist of resistin.

19. The method according to claim 18, wherein the antagonist is an antibody to resistin or a compound that interferes with the binding of resistin to its receptor.

20. The method according to claim 12, wherein said disorder is atherosclerosis.


SEQUENCE LISTING (6 sequences)

SEQ ID NO: 1 - DNA, 21 bp, Artificial
  Sense-Resistin
agccatcaatgataggatcca

SEQ ID NO: 2 - DNA, 20 bp, Artificial
  Antisense-Resistin
tccaggccaatgctgcttat

SEQ ID NO: 3 - DNA, 27 bp, Artificial
  Resistin Probe
aggtcgccggctccctaatatttaggg

SEQ ID NO: 4 - DNA, 22 bp, Artificial
  Sense human 36B4 sense
tcgtggaagtgacatcgtcttt

SEQ ID NO: 5 - DNA, 19 bp, Artificial
  Antisense 36B4
ctgtcttccctgggcatca

SEQ ID NO: 6 - DNA, 20 bp, Artificial
  36B4 Probe
tggcaatccctgacgcaccg